(19) United States
(12) Patent Application Publication
(10) Pub. No.: US 2026/0011025 A1
(43) Pub. Date: Jan. 8, 2026

(54) PARALLEL LASER LINE SCANNING DEVICE AND METHOD FOR MEASURING CRACK WIDTH AND SPALLING AREA

(71) Applicant: The University of Hong Kong, Hong Kong (HK)

(72) Inventors: Kai Leung SU, Hong Kong (HK); Chaobin LI, Hong Kong (HK)

(21) Appl. No.: 19/250,138

(22) Filed: Jun. 26, 2025

(30) Foreign Application Priority Data
Jul. 2, 2024 (HK) 32024093550.4

(51) Int. Cl. G06T 7/60; G01B 11/02; G01N 21/88; G01N 33/38; G06T 7/00; G06T 7/13; G06V 10/25; G06V 10/28; G06V 10/82

(52) U.S. Cl. CPC G06T 7/60; G01B 11/02; G01N 21/8851; G01N 33/383; G06T 7/0002; G06T 7/13; G06V 10/25; G06V 10/28; G06V 10/82; G06T 2207/20084; G06T 2207/30184

(57) ABSTRACT

The application discloses a parallel laser line scanning device and a method for measuring crack width and spalling area. The device includes a laser source configured to emit two beams of laser light in parallel to each other to scan a surface; a camera configured to collect an image of the scanned surface, and the camera comprising a lens and an image sensor; a positioning rod; and a controller configured to process an image of scanned surface to obtain a pixel scale of a crack or three-dimensional coordinates of contour points of a spalling, and determine an actual crack width in the scanned surface according to the pixel scale of the crack, or determining an actual spalling area according to the three-dimensional coordinates of the contour points of the spalling. The device facilitates faster and more objective evaluations of building conditions, contributing to improved structural health monitoring and maintenance decision-making.

### CROSS-REFERENCE TO RELATED APPLICATIONS

The present application claims priority from the Hong Kong Short Term patent Application No. 32024093550.4 filed on Jul. 2, 2024, and the disclosure of which is incorporated herein by reference in its entirety.

### FIELD OF THE INVENTION

The present application relates to the field of measurement technology, in particular to a parallel laser line scanning device and a measurement method for surface crack width and spalling area.

### BACKGROUND OF THE INVENTION

With the increase of service time, due to the low tensile strength of concrete, cracks and spalling will inevitably occur in concrete structures under the influence of external loads and temperature changes. The presence of cracks and spalling can adversely affect the deterioration of the building, for example, it can accelerate the corrosion of steel bars and seriously impair the structural integrity and durability of concrete buildings (Su, R. K. L., Zhang, Y. L.: a novel elastic-body-rotation model for concrete cover spalling caused by non-uniform corrosion of reinforcement. Construction and Building Materials, 213, 549-560 (2019)). Given that crack and spalling are key indicators of a structure's condition and longevity, crack and spalling detection is of considerable importance in the maintenance of concrete structures. Traditional crack and spalling detection methods rely on regular manual inspections, which typically involve inspectors evaluating defects using a variety of measurement tools, such as crack scales. However, the results are susceptible to subjective factors, and the process is time-consuming, labor-intensive, and sometimes dangerous (Lee, B. Y., Kim, Y. Y.: Automated image processing technique for detecting and analysing concrete surface cracks. Struct. Infrastruct. Eng. 9, 567-577 (2013)). In addition, hard-to-access locations are inspected based on the inspector's experience, which means that subjective assessments may lack reliability. Therefore, the research on high-precision, high-efficiency non-contact crack and spalling detection plays a crucial role in effective defect management.

In order to overcome the shortcomings of manual detection methods, many crack detection methods based on image processing technology have been proposed over the years, such as image thresholding (Fujita, Y., Hamamoto, Y.: A robust automatic crack detection method from noisy concrete surfaces. Mach. Vision. Appl. 22 (2): 245-254 (2010)), (Oliveira, H., Correia, P. L.: Automatic road crack segmentation using entropy and image dynamic thresholding. In: 2009 17th European signal processing conference, Glasgow, Scotland, pp. 622-626 (2009)), edge detection (Canny, J.: A Computational Approach to Edge Detection. IEEE Trans. Pattern Anal. Mach. Intell. 6, 679-698 (1986)), (Abdel-Qader, I., Abudayyeh, O., Kelly, M. E.: Analysis of edge-detection techniques for crack identification in bridges. J. Comput. Civ. Eng. 17 (4), 255-263 (2003)) and morphological operation methods (Merazi-Meksen, T., Boudraa, M, Boudraa, B.: Mathematical morphology for TOFD image analysis and automatic crack detection. Ultrasonics, 54 (6), 1642-1648 (2014)), (Giakoumis, I., Nikolaidis, N., Pitas, I.: Digital image processing techniques for the detection and removal of cracks in digitized paintings. IEEE Trans Image Process 2006, 15 (1), 178-188 (2006)). Although these methods are useful, they rely heavily on manually set parameters and are inefficient in complex environments. To this end, deep learning has emerged as a superior alternative that can autonomously extract detailed features from large amounts of crack and spalling data (Cha, Y.-J., Choi, W., Büyüköztürk, O.: Deep Learning-Based Crack Damage Detection Using Convolutional Neural Networks. Comput. Civ. Infrastruct. Eng. 32 (5), 361-378 (2017)). Deep learning, especially semantic segmentation methods that classify individual pixels in an image (Rao, A. S., Nguyen, T., Palaniswami, M.: Vision-based automated crack detection using convolutional neural networks for condition assessment of infrastructure. Struct. Health. Monit. 20 (4), 2124-2142 (2021)) significantly improved crack and spalling detection accuracy and efficiency. Benefited from the latest image classification architecture (Ronneberger, O., Fischer, P., Brox, T.: U-Net: Convolutional Networks for Biomedical Image Segmentation. arXiv (2015). preprint arXiv: 1505.04597), (Dung, C. V.: Autonomous concrete crack detection using deep fully convolutional neural network. Autom. Constr. 99, 52-58 (2019)), these methods apply advanced encoder-decoder models. Studies have shown that the U-Net semantic segmentation model, which is known for its sensitivity to edge details, is suitable for concrete defect segmentation (Zhang, L., Shen, J., Zhu, B.: A research on an improved Unet-based concrete crack detection algorithm. Struct. Health Monit. 20 (4), 1864-1879 (2020)).

Once a crack is detected from the image, an assessment of the crack (e.g., crack width and length) through direct needs can help the user in decision-making and further maintenance planning (AASHTO: Guide Manual for Bridge Element Inspection. Bridge Element Inspection Manual, p. 172 (2011)). However, there are some challenges in the process of capturing crack images to quantifying physical dimensions. Traditional methods typically use a single camera and require a physical scale or marker attached to the concrete surface to convert the pixel information into actual measurements. Although this method is widely used, it is very cumbersome and often impractical under complex field conditions (Yamaguchi, T., Hashimoto. S.: Practical image measurement of crack width for real concrete structure. Electronics & Communications in Japan, 92 (10), 1-12 (2010) DOI: 10.1002/ecj.10151). There is also the option of calculating the distance between the camera and the crack to establish an accurate pixel scale. However, this method requires the camera to be completely perpendicular to the crack surface, and failure to meet this requirement can lead to significant errors in measurement accuracy (Yoon, J., Shin, H., Song, M.: A Crack Width Measurement Method of UAV Images Using High-Resolution Algorithms. Sustainability, 15 (2022). DOI: 10.3390/su15010478). There are also researchers (Zhao, S. Z., Kang, F., Li., J. J.: Non-Contact Crack Visual Measurement System Combining Improved U-Net Algorithm and Canny Edge Detection Method with Laser Rangefinder and Camera. Applied Sciences-Basel, 12 (20), 10651 (2022)) explores the use of a laser rangefinder to calculate the pixel scale to measure crack width. Although this technique improves measurement accuracy, it does not provide automatic measurement capabilities, limiting its practical application. To overcome these limitations, advanced computer vision techniques have been developed. For example, stereo vision systems (Shan, B., Zheng, S., Ou, J.: A stereovision-based crack width detection approach for concrete surface assessment. KSCE Journal of Civil Engineering (2016). DOI: 10.1007/s12205-015-0461-6) and Light Detection and Ranging (LiDAR) technology (Yang, H., Xu, X.: Intelligent crack extraction based on terrestrial laser scanning measurement. London, Institute of Measurement and Control, 53:002029401987749 (2020). DOI: 10.1177/0020294019877490) is used to derive the exact three-dimensional coordinates of the crack. These complex methods facilitate a more detailed analysis of the crack geometry, but are more complex and costly.

While existing studies have largely focused on the estimation of wide cracks, recent studies have shown that quantitatively assessing small cracks presents significant challenges. It is found that the image must contain at least five pixels of the crack width in order to maintain a low error rate when measuring the crack width (Ni, F. T., Zhang, J., Chen, Z. Q.: Zernike-moment measurement of thin-crack width in images enabled by dual-scale deep learning. Computer Aided Civil & Infrastructure Engineering (2018). DOI: 10.1111/mice.12421). To achieve this level of detail, it is often necessary to capture images at close range. To address the difficulty of measuring small cracks, other research avenues have explored the use of high-resolution algorithms to improve the quality of degraded images, especially those used to determine crack widths (Yoon, J., Shin, H., Song, M.: A Crack Width Measurement Method of UAV Images Using High-Resolution Algorithms. Sustainability, 15 (2022) DOI: 10.3390/su15010478). Although the well-known Canny approach to edge detection (Canny, J.: A Computational Approach to Edge Detection. IEEE Trans. Pattern Anal. Mach. Intell. 6, 679-698 (1986)) can delineate the boundaries of cracks at the pixel level, but it is not sufficient to measure small cracks due to limited resolution. In this regard, some researchers proposed a new algorithm combining Canny edge detection with Zernike moment (Shan, B. H., Zheng, S. J., Ou, J. P.: A stereovision-based crack width detection approach for concrete surface assessment. KSCE Journal of Civil Engineering (2016). DOI: 10.1007/s12205-015-0461-6) to measure three-dimensional cracks, achieving an accuracy of up to 0.02 subpixels when identifying crack edges. Still, the accuracy of this method is susceptible to interference from ambient lighting conditions.

While deep learning algorithms can automatically identify spalling regions from images, they cannot quantify their physical attributes, such as spalling area critical for structural evaluation and repair prioritization. To compensate for this shortcoming, some studies have attempted to combine spalling segmentation results with three-dimensional measurement techniques such as photogrammetry, laser triangulation, and light detection and technology (LiDAR). These systems generate point cloud data (PCD) that can be used to approximate the geometry of the concrete surface. For example, Kim et al. (Kim, M.-K., Sohn, H., & Chang, C.-C. Localization and quantification of concrete spalling defects using terrestrial laser scanning. Journal of Computing in Civil Engineering 29 (6), 04014086 (2015)) uses laser scanning to detect spalling on concrete panels, while Rau et al. (Rau, J. Y., Hsiao, K., Jhan, J. P., Wang, S. C., Fang, W. T., & Wang, J. Concrete spalling volume estimation using three-dimensional reconstructed surface model. Remote Sensing, 9 (4), 356 (2017)) used grid reconstruction to estimate the volume of concrete loss. Despite the potential of these three-dimensional methods, there are still several challenges in the practical application of spalling area calculation: (1) extracting spalling geometric information from point clouds is computationally expensive, often requires manual intervention or relies on complex mesh reconstruction algorithms (; Tang, P., Huber, D., Akinci, B., Lipman, R., & Lytle, A. Automatic reconstruction of as-built building information models from laser-scanned point clouds: A review of related techniques. Automation in Construction, 19 (7), 829-843 (2010)); (2) Outdoor deployment systems are susceptible to noise, and occlusion (such as shadows, surface stains, etc.) can interfere with accurate surface reconstruction (Xu, Y., Chen, S., & Chen, Y. A comprehensive review of three-dimensional reconstruction techniques in civil engineering. Archives of Computational Methods in Engineering, 28, 1431-1457 (2021)); (3) The data processing process is time-consuming, and it is difficult to meet the efficiency requirements of daily large-scale inspections (Adán, A., Quintana, B., Pricto, S. A., & Bosché, F. Scan-to-BIM for energy performance analysis: Experiences from a real-world project. Automation in Construction, 84, 488-500 (2017)).

### SUMMARY OF THE INVENTION

Therefore, the provision of a new parallel laser line scanning device and its measurement method for crack width and spalling area in the present application can improve the accuracy and efficiency of the measurement of building surface defects (cracks, spalling).

According to the first aspect of the embodiment of the present application, a parallel laser line scanning device is provided and comprising: a laser source configured to emit two beams of laser light in parallel to each other to scan a surface; a camera configured to collect an image of the scanned surface, and the camera comprising a lens and an image sensor; a positioning rod, with the camera being arranged at a first end of the positioning rod and the laser source being arranged at a second end of the positioning rod; and a controller configured to process an image of an scanned surface to obtain a pixel scale of a crack or three-dimensional coordinates of contour points of a spalling, and determine an actual crack width in the scanned surface according to the pixel scale of the crack, or determining an actual spalling area according to the three-dimensional coordinates of the contour points of the spalling.

Preferably, the laser source is a two-line laser diode or comprises two lasers.

Preferably, the controller is further configured to perform the following steps: utilizing a pre-trained neural network model to identify a crack region image or a spalling region image from the image of the scanned surface; determining subpixel crack width and direction of the crack according to the crack region image; recognizing a laser fringe region image from the image of the scanned surface, determining the pixel scale of the crack or the three-dimensional coordinates of the contour points of the spalling according to the laser fringe region image and the identified crack or spalling region image; determining the actual crack width according to the pixel scale of the crack, the subpixel crack width and the direction of the crack; and determining the actual spalling area according to the three-dimensional coordinates of the contour points of the spalling.

Preferably, the neural network adopts a U-Net image segmentation network model.

Preferably, determining the subpixel crack width according to the crack region image comprises: converting the crack region image into a single-channel grayscale crack image; converting the single-channel grayscale crack image into a three-dimensional grayscale image, and fitting background light intensity in the three-dimensional grayscale image to obtain a background image; subtracting the single-channel grayscale image from the background image to obtain a background-reduced crack image; and calculating subpixel crack width of each pixel row in the background-reduced crack image by using the equivalent area principle.

Preferably, the background light intensity at the crack pixels in the three-dimensional grayscale image is fitted using RANSAC algorithm to obtain the background image.

Preferably, determining the pixel scale of the crack or the three-dimensional coordinates of the contour points of the spalling according to the laser fringe region image and the identified crack region image or spalling region image comprises: calibrating system parameters of the parallel laser line scanning device; determining a laser band center according to the laser fringe region image; fitting two parallel laser baselines according to the laser band center; constructing a three-dimensional measurement model representing relative positional relationship between laser spots on the scanned surface and image pixels in the image sensor; generating two sets of laser point clouds corresponding to the two laser baselines in the laser fringe region image based on the three-dimensional measurement model, and determining a target plane based on the laser point clouds and the system parameters; determining the three-dimensional coordinates of contour points of the crack or spalling according to the target plane and pixel coordinates of contour points of the identified crack or spalling; determining a pixel proportion of the crack according to the pixel coordinates and three-dimensional coordinates of the crack.

Preferably, the Steger method is used to determine the laser band center according to the image of the laser fringe region.

Preferably, the RANSAC method is used to fit the two parallel laser baselines according to the laser band center.

Preferably, determining the three-dimensional coordinates of contour points of the crack or spalling according to the target plane and the pixel coordinates of contour points of the identified crack or spalling comprises: calibrating system parameters of the parallel laser line scanning device; and constructing a three-dimensional measurement model for representing a relative positional relationship between object points on the target plane and image pixels in the image sensor.

Preferably, determining the direction of the crack according to the crack region image and the identified crack region comprises: converting the crack region image into a binary image; using a skeletonization technique to extract a center line of the crack from the binary image to obtain a crack skeleton; and analyzing a direction of the skeleton according to the crack skeleton to determine the direction of the crack.

Preferably, determining the crack width according to the pixel scale, the subpixel crack width and the direction of the crack comprises: locating a subpixel-level width of the crack on each pixel row perpendicular to the direction of the crack according to the direction of the crack and the subpixel crack width of each pixel row; and determining the actual crack width according to the subpixel-level width of the crack located on each pixel row perpendicular to the direction of the crack and the pixel scale.

Preferably, determining the actual spalling area according to the three-dimensional coordinates of the contour points of the spalling comprises: performing coordinate conversion of the three-dimensional coordinates of the contour points of the spalling to obtain a two-dimensional coordinate point set on the target plane; sorting the set of two-dimensional coordinate points on the target plane according to a clockwise closed path; using the Socrace algorithm to process the set of two-dimensional coordinate points on the target plane, automatically adapting the uneven contour to determine the actual spalling area.

According to the second aspect of the embodiment of the present application, a method for measuring crack width of a crack or spalling area of a spalling using the parallel laser scanning device in the first aspect is provided, the method comprising: using the laser source to emit two laser beams in parallel to each other to scan a surface; using the camera to capture an image of the scanned surface with a camera; and using the controller to process the captured image of the scanned surface to determine the actual crack width or the actual spalling area in the scanned surface.

Preferably, processing the captured image of the scanned surface to determine the crack width in the scanned surface comprises: using a pre-trained neural network to identify a crack region image from the image of the scanned surface; identifying a laser fringe region image from the image of the scanned surface, determining the pixel scale of the crack according to the laser fringe region image and the identified crack region image; determining subpixel crack width and direction of the crack according to the crack region image; and determining the actual crack width according to the pixel scale, the subpixel crack width and the direction of the crack.

Preferably, processing the captured image of the scanned surface to determine the spalling area in the scanned surface comprises: using a pre-trained neural network to identify the spalling region image from the image of the scanned surface; identifying a laser fringe region image from the image of the scanned surface, determining three-dimensional coordinates of contour points of the spalling according to the laser fringe region image and the identified spalling region image; and determining the actual spalling area according to the three-dimensional coordinates of the contour points of the spalling.

Compared with the prior art, the application can accurately extract the pixel scale data of the crack or the three-dimensional coordinate data of the contour points of the spalling from the image of the scanned surface, and determine the crack width or the spalling area in the scanned surface according to the pixel scale of the crack or the three-dimensional coordinates of the contour points of the spalling, thereby minimizing the problem of pixel distortion common in non-vertical photography. As a result, the accuracy and efficiency of the measurement of cracks and spalls in out-of-plane buildings are improved conducive to a more objective and rapid assessment of the building condition.

### BRIEF DESCRIPTION OF THE DRAWINGS

The embodiments of the present application are further described below with reference to the accompanying drawings, wherein:

FIG. 1 is a schematic diagram of vertical and non-vertical photography;

FIGS. 2A and 2B are schematic diagrams of use scenarios of a parallel laser line scanning device in crack and spalling measurements according to one embodiment of the present application, respectively;

FIG. 3 is a flowchart of the processing of the image of the scanned surface according to one embodiment of the present application;

FIG. 4 is a flowchart of the process of measuring crack width in the image of the scanned surface according to one embodiment of the present application;

FIG. 5 is a schematic diagram of the process of measuring cracks according to one embodiment of the present application;

FIG. 6 is a schematic diagram of the U-Net network structure for crack segmentation according to one embodiment of the present application;

FIG. 7 is a flowchart of the process for measuring the spalling area in the image of the scanned surface according to one embodiment of the present application;

FIG. 8 is a schematic diagram of the U-Net network structure used for spalling segmentation according to one embodiment of the present application;

FIG. 9 is a schematic scene in which an artificial crack or spalling is measured by a parallel laser line scanning device according to one embodiment of the present application;

FIG. 10A shows the measurement results of the distance between adjacent corner points over a range of 1.0 m to 2.0 m using vertical photogrammetry;

FIG. 10B shows the measurement of the distance between adjacent corner points at different angles at a measurement distance of 2.0 m.

FIG. 11A and FIG. 11B show the measurement results of various artificial cracks using vertical photography at a measurement distance of 1.0 m;

FIG. 12 shows the average measurement schematic results and associated schematic errors for artificial cracks with widths of 1.5 mm, 1.0 mm, 0.75 mm, 0.5 mm, and 0.25 mm using vertical photography over a measurement distance range of 1.0 m to 2.0 m;

FIG. 13 shows the average schematic results and schematic error of the measurement of artificial cracks with widths of 1.5 mm, 1.0 mm, and 0.5 mm at a measurement distance of 1.5 m;

FIG. 14 is a schematic diagram of the influence of different lighting conditions on the measurement results of artificial cracks with widths of 1.5 mm, 1.0 mm and 0.5 mm at a measurement distance of 1.5 m by using vertical photography according to the embodiment of the present application;

FIG. 15A is a schematic diagram of the training and validation intersection ratio curve of the U-Net model used for segmenting cracks according to the embodiment of the present application;

FIG. 15B is a schematic diagram of the performance of the U-Net model for segmenting cracks by means of a confusion matrix according to the embodiment of the present application;

FIG. 16 is a schematic diagram of the complete process of crack width measurement from crack segmentation to skeleton generation, direction generation and width determination according to the embodiment of the present application;

FIG. 17A to FIG. 17I show the results of the measurements of three types of cracks (Crack 1, Crack 2 and Crack 3) at measurement distances of 1.0 m, 1.5 m and 2.0 m using vertical photography;

FIG. 18 shows the average crack width of Crack 1, Crack 2, and Crack 3 measured using vertical photography over a measurement distance range of 1.0 m to 2.0 m;

FIG. 19 shows the average crack width measurement results of Crack 1, Crack 2 and Crack 3 with a measurement distance of 1.5 m and a measurement angle range of 45° to 135°;

FIG. 20 shows the average crack width measurements of cracks 1, 2 and 3 at a measuring distance of 1.5 m under different ambient illuminations.

FIGS. 21A and 21B show the raw image and segmentation results of area measurement sample I, respectively;

FIGS. 22A and 22B show the raw images and segmentation results of area measurement sample II, respectively;

FIGS. 23A and 23B show the area measurements for samples I and II, respectively;

FIG. 24A is a schematic diagram of the training and validation intersection ratio curve of the U-Net model used for segmentation and spalling according to the embodiment of the present application;

FIG. 24B is a schematic diagram showing the performance of the U-Net model for segmentation spalling by means of a confusion matrix according to the embodiment of the present application

FIGS. 25A and 25B show images and segmentation results of a sample of the exfoliated area, respectively;

FIG. 26 shows measuring the on-site scan scene of the soffit of the parking lot ceiling;

FIGS. 27A and 27B show the raw images and segmentation of Sample III scanned under brighter lighting conditions (1200 lux), respectively;

FIGS. 28A and 28B show the raw image and segmentation of sample IV scanned under darker lighting conditions (150 lux), respectively;

FIGS. 29A and 29B show the area measurements for samples III and IV, respectively.

### DETAILED DESCRIPTION

In order to make the purpose of the present application, the technical solution and its advantages more clearly understood, the present application is further described in detail below by means of specific embodiments in conjunction with the accompanying drawings. It should be understood that the specific embodiments described herein are only used to interpret the present application and are not intended to qualify the present application.

In order to better understand the present application, the measurement principle and the problems encountered in the application of the existing crack measurement or identification technology are first described based on the camera pinhole model.

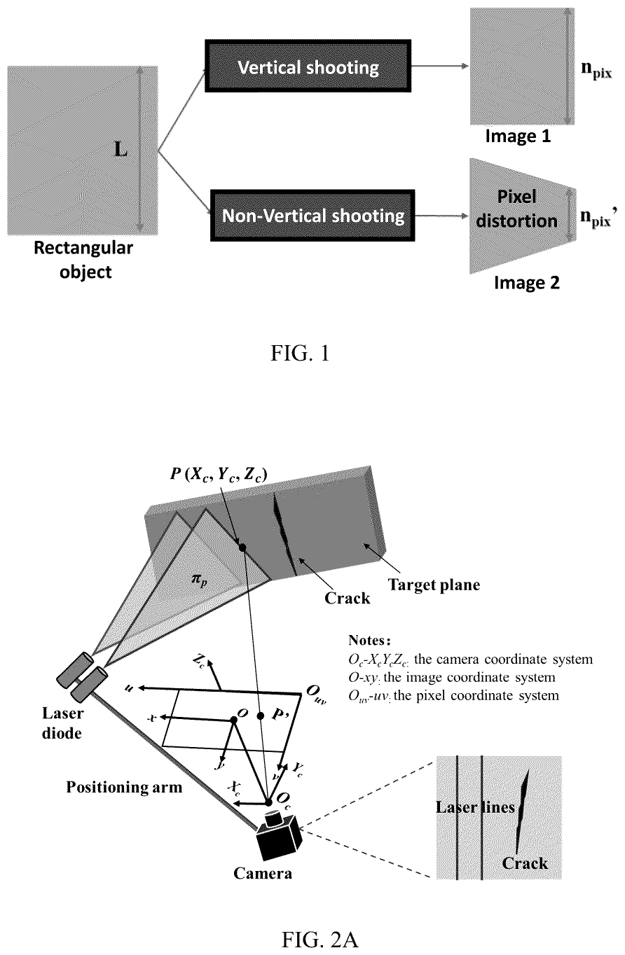

As shown in FIG. 1, the target plane is set to be parallel to the image plane of the camera to ensure vertical shooting without distortion. The length L of the object on the target plane is projected onto the image plane as a series of image pixels (the total number of pixels is denoted asnₚᵢₓ the nₚᵢₓ total number of pixels in FIG. 1 and is specifically represented as the total number of pixels in image 1). The relationship between the length L of an object and the pixels of the image projected onto the image plane can be defined as Eq. (1).

$$L = \frac{f}{D} \times s \times {n}_{pix} . \qquad ( 1 )$$

where D represents the measurement distance, f represents the focal length of the camera, and s represents the actual size of the image pixels,

$$\frac{f}{D} \times s$$

which represents the basic form of pixel proportion, i.e., the ratio of the representation of an object in a photograph to its actual size, through which the actual length of each pixel can be determined (e.g., each pixel corresponds to 0.1 mm).

However, Eq. (1) faces three challenges in practical application:
1) Accurate Measurement Distance: Theoretically, the measurement distance is the distance from the center of the camera lens to the surface of an object. Determining this distance accurately is challenging, and it is easy to introduce errors into Eq. (1).
2) Camera Parameters and Image Distortion: Applying accurate camera parameters is essential for the correct application of Eq. (1). In addition, the camera pinhole model is an idealized linear imaging model that does not account for the nonlinear distortions caused by manufacturing and installation errors that must be considered in practical applications.
3) Non-perpendicular photography: In real life, the image plane of the camera is usually not parallel to the target plane, resulting in non-perpendicular photography. As shown in FIG. 1, non-perpendicular photography results in different measurement distances from different parts of the object to the camera lens, resulting in pixel distortion. The total number of pixels expressed as image 2. Because it is difficult to obtain accurate pixel scales at every position in the image, correcting the distorted object image to its actual size is a challenge.nₚᵢₓ′

The classical Canny edge detection algorithm is widely used in crack identification, and its principle is to distinguish edges by the change of local gray value. Canny's algorithm is an objective mathematical method that calculates the gradients Pₓ[i, j] and P_{y}[i, j] in the x and y directions by the first-order finite difference shown in Eq. (2) and Eq. (3), and identifies the edges based on the gradients.

$${P}_{x} [ i , j ] = \frac{I [ i , j + 1 ] - I [ i , j ] + I [ i + 1 , j + 1 ] - I [ i + 1 , j ] ]}{2} \qquad ( 2 )$$

$${P}_{y} [ i , j ] = \frac{I [ i , j ] - I [ i + 1 , j ] + I [ i , j + 1 ] - I [ i + 1 , j + 1 ] ]}{2} \qquad ( 3 )$$

where i and j are the coordinate positions of each pixel, and I is the pixel value.

The amplitude of the pixel M[i, j] can be calculated according to Eq. (4):

$$M [ i , j ] = \sqrt{{{P}_{x} [ i , j ]}^{2} + {{P}_{y} [ i , j ]}^{2}} \qquad ( 4 )$$

The orientation of the pixel θ[i, j] can be calculated according to Eq. (5):

$$θ [ i , j ] = arctan ( \frac{{P}_{y} [ i , j ]}{{P}_{x} [ i , j ]} ) \qquad ( 5 )$$

However, in real-world scenes, if a tiny crack is not prominent enough in the overall image, the Canny algorithm may mistake the non-cracked edge for the crack, and cause the detected boundary results to be discontinuous due to the blurred crack edge. As a result, the results may become noisy and discontinuous with background noise. In addition, the Canny algorithm can only measure crack width at the pixel level, which may introduce inaccuracies in the presence of tiny cracks, especially when taking images at a long distance (Aslam, Y., Santhi, N., Ramasamy, N., Ramar, K.: Localization and segmentation of metal cracks using deep learning. J. Ambient Intell. Humaniz. Comput. 12, 4205-4213 (2021)), (Wang, H. F., Zhai, L., Huang, H.: Measurement for cracks at the bottom of bridges based on tethered creeping unmanned aerial vehicle. Autom. Constr. 2020, 119, 103330 (2020)).

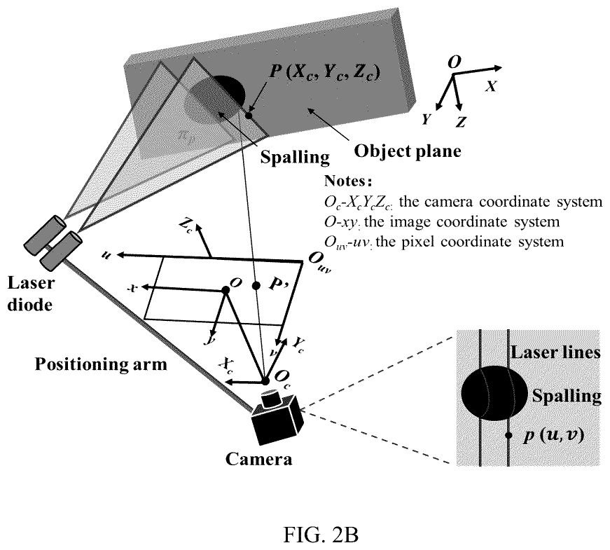

FIGS. 2A and 2B respectively show schematic diagrams of the scene of measuring cracks and spalls using a parallel laser line scanning device according to one embodiment of the present application. The parallel laser line scanning device mainly comprises a laser source, a digital camera, a positioning rod, and a controller (not shown). The laser source is configured to emit two laser beams in parallel to each other to scan a surface. A digital camera (also known as a camera for short) is configured to capture an image of the scanned surface. The positioning rod is used to mount or couple the laser source and the digital camera. The controller is configured to determine the crack width or the spalling area in the scanned surface by processing the captured image of the scanned surface. Wherein, the laser source that can emit two parallel laser lines can be a two-line laser diode or a laser source composed of two lasers. The digital camera includes at least a lens and an image sensor. The digital camera is attached to the first end of the positioning rod by a coupling mechanism, while the laser source is attached to the second end of the positioning rod. The positioning rod is a retractable metal rod or metal arm, the length of which can be adjusted according to the actual measurement scenario. During the measurement, the digital camera is focused to the scanned surface, and the horizontal direction on which it is located is parallel to the measurement reference surface, while the positioning rod is inclined at an angle relative to the horizontal direction where the digital camera is located, and this angle can be adjusted by the coupling mechanism between the digital camera and the positioning rod. The laser source projects parallel laser lines onto the scanned surface at certain angle. The digital camera and laser source can be self-poared, making it easy to use at outdoors.

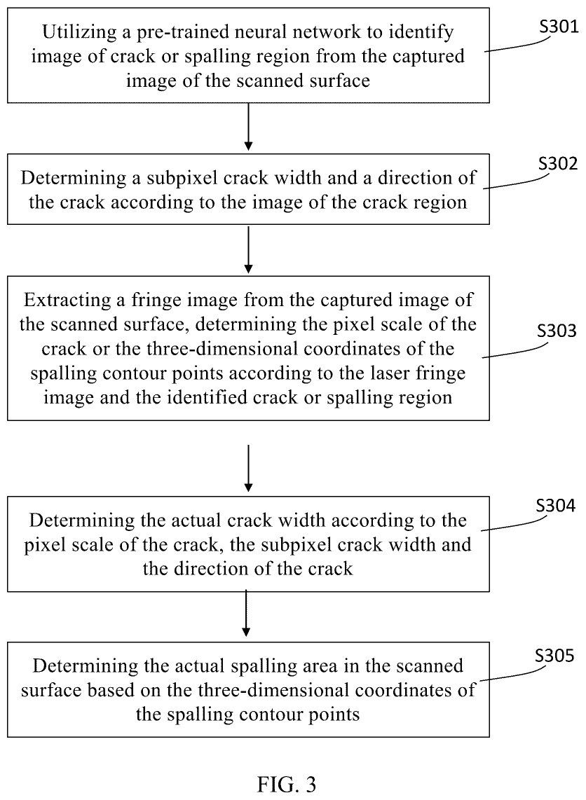

In the parallel laser line scanning device shown in FIGS. 2A and 2B, the image of the scanned surface captured by the camera contains laser fringes and cracks or spalls to be measured. The processing of the captured image of the scanned surface is performed by the controller. In one embodiment of the present application, the controller is configured to perform the steps shown in FIG. 3, comprising: step S301, utilizing a pre-trained neural network to identify image of crack or spalling region from the captured image of the scanned surface; step S302, determining a subpixel crack width and a direction of the crack according to the image of the crack region; step S303, extracting an image of laser fringe region (subsequently referred to as laser fringe image) from the captured image of the scanned surface, determining the pixel scale of the crack or the three-dimensional coordinates of the contour points of the spalling according to the laser fringe image and the identified crack or spalling region; step S304, determining the actual crack width according to the pixel scale of the crack, the subpixel crack width and the direction of the crack; step S305, determining the actual spalling area in the scanned surface based on the three-dimensional coordinates of the contour points of the spalling.

In another embodiment of the present application, a method for measuring crack width or spalling area using the parallel laser scanning device described above is also provided. The method mainly comprises: step 1) using a laser source to emit two laser beams in parallel to each other to scan a surface containing cracks or spalling to be measured; step 2) using a camera to capture an image of the scanned surface containing parallel lines of laser projection on the scanned surface and any cracks or spalls that may exist on the surface; step 3) The captured image of the scanned surface is processed to determine the crack width or the spalling area in the scanned surface. Wherein, step 3) comprises using a pre-trained neural network to identify the crack or spalling region image from the image of the scanned surface, and determine the subpixel crack width and direction of the crack based on the crack region image; identify the image of the laser fringe region from the image of the scanned surface, and determine the pixel scale of the crack or the three-dimensional coordinates of the contour points of the spalling according to the laser fringe image and the identified crack or spalling region; then, the actual crack width is determined according to the pixel scale, sub-pixel crack width and direction of the crack; and the actual spalling area in the scanned surface is determined based on the three-dimensional coordinates of the contour points of the spalling.

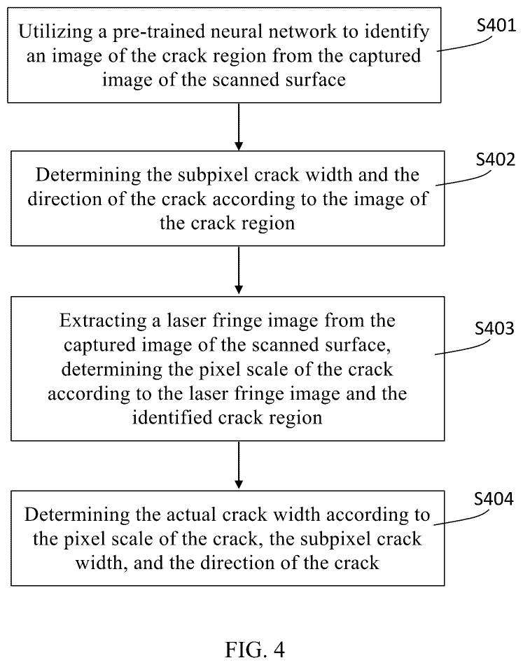

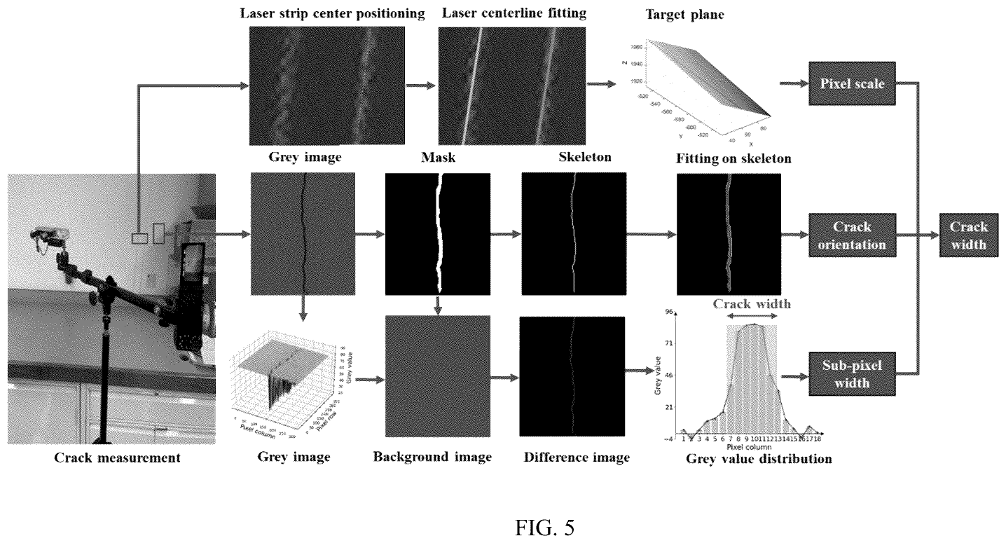

In another embodiment of the present application, the controller is configured to perform steps as shown in FIG. 4 to measure the crack width, comprising: step S401, utilizing a pre-trained neural network to identify an image of the crack region from the captured image of the scanned surface; step S402, determining the subpixel crack width and the direction of the crack according to the image of the crack region; step S403, extracting a laser fringe region image (subsequently referred to as a laser fringe image) from the captured image of the scanned surface, determining the pixel scale of the crack according to the laser fringe image and the identified crack region; step S404, determining the actual crack width according to the pixel scale of the crack, the subpixel crack width, and the direction of the crack. The above steps are described in detail below according to the process of measuring cracks shown in FIG. 5.

In step S401, the crack region is identified.

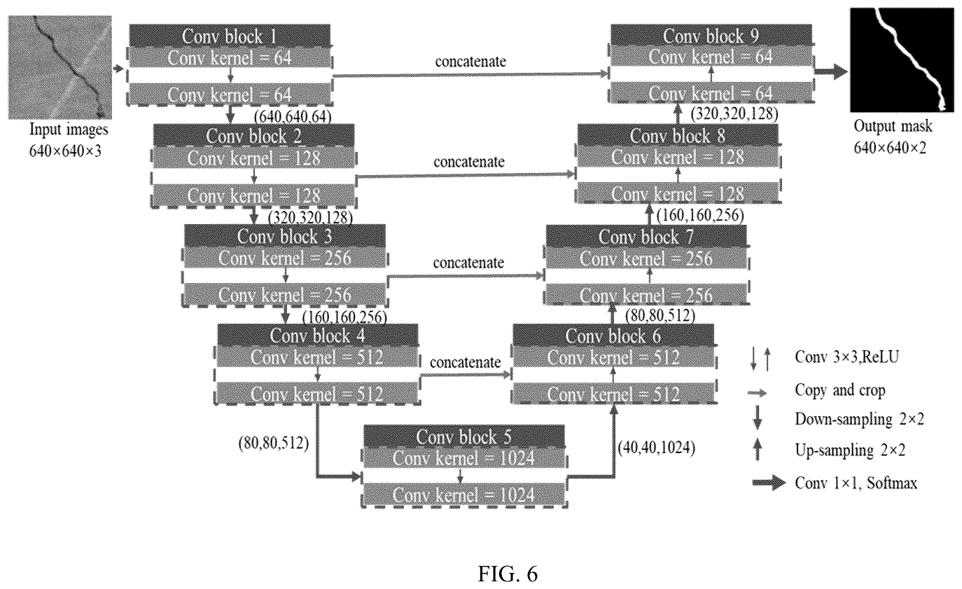

In one embodiment, a pre-trained neural network (which may be called an image segmentation network) is utilized to automatically segment or identify an image of a crack region from the captured image of the scanned surface. As shown in FIG. 6, in this embodiment, the U-Net network model is used to identify the image of the crack region. In another embodiment, the backbone of the U-Net network model can also be replaced with ResNet-50. ResNet-50 is a convolutional neural network architecture known for its deeper structures and hopping connections, resulting in better feature extraction and information flow. Such model modifications enhance the capabilities of the segmentation process and contribute to more accurate and robust results.

In the present application, the use of the U-Net model for crack identification is used as an example. The input of the U-Net model is an RGB crack image, and the output is a binary image of semantic segmentation, in which the area of the crack is obtained by the U-Net algorithm. The architecture of the U-Net model is shown in FIG. 6 and consists of an input layer, an output layer, and an intermediate layer. The middle layer consists of multiple convolution blocks (Conv blocks), each of which contains two convolutional layers. In FIG. 6, the left part represents the shrinking path, and the right part represents the expansion path. When the input RGB crack image of size 512×512 passes through the shrinkage path, the middle layer extracts the high-resolution features in the crack image. After the output layer and the first convolutional layer of each convolutional module, the RGB crack image is compressed into a multi-channel feature map using 3×3 cores, and then the activation function is used to add nonlinearity to the neural network to enable it to fit more nonlinear functions. In one implementation, the ReLU activation function a type of activation function commonly used to enhance nonlinear properties, is used. In order to reduce the number of parameters and the risk of overfitting in the network, the down-sampling operation is used as an intermediate process to connect each convolution module in the shrinkage path. Wherein, the maximum pooling operation is one of the methods of down-sampling, which has been shown to be able to preserve features well. After passing through the encoding procedure, the feature is passed to the extended path on the right in FIG. 6. In contrast to the shrink path, the extended path progressively expands the calculated data shape to the original input image size to precisely restore and locate features. At the same time, the parallel feature maps from the shrinking path are passed directly to the extended path via the connection. The output layer is designed to be 1×1 in size and is used to convert the output image to binary of the same size and will store the category of each pixel into that pixel. In order to compress the pixel classification results in the form of normalization, the Softmax function is used in the output layer for normalization.

The loss function is the primary metric that describes the deviation between the prediction generated by the network and the true value. The purpose of training the model is to minimize the loss value and make the final segmentation more accurate. In previous studies on crack semantic segmentation, several classical loss functions have been widely adopted, including cross-entropy loss function, dice loss function, focus loss function and their various combinations (Aslam, Y., Santhi, N., Ramasamy, N., Ramar, K.: Localization and segmentation of metal cracks using deep learning. J. Ambient Intell. Humaniz. Comput. 12, 4205-4213 (2021)). In addition, data imbalance is a problem in crack identification tasks. Due to the small proportion of crack pixels, the training weight may be offset. In order to obtain the accuracy loss value in the training process and solve the problem of data imbalance, the combination of Generalized Dice Loss (GDL) and Cross-Entropy Loss (CEL) is selected as the loss function.

After processing by the U-Net algorithm, the crack regions in each image can be identified and extracted. The quality of the trained model is usually evaluated by three parameters: precision, recall rate, and cross-union ratio IoU:

$$Precision = \frac{T P}{T P + F {P}^{′}} \qquad ( 8 )$$

$$Recall = \frac{T P}{T P + F {N}^{′}} \qquad ( 9 )$$

$$IoU = \frac{T P}{T P + F {P}^{′} + F {N}^{′}} \qquad ( 10 )$$

where TP is true positive, indicating the number of true positive pixels; FP is false positive, indicating the number of false-positive pixels. FN is false negative, indicating the number of false-negative pixels.

In step S402, the subpixel crack width and direction of the crack are determined based on the crack region image:

Due to the imperfection of the model and the fact that the dataset used for training may not be accurate, the crack edges generated by the U-Net model are not always accurate, especially in the case of microcrack measurements. As a result, the crack edge needs to be further refined. In fact, for micro-crack images, the boundary between the background and the crack is usually not obvious, and the transition region of the grayscale gradient is only a few pixels wide, which often leads to misjudgment of the crack boundary. Therefore, in one embodiment, a method for determining width of the crack at subpixel level: equal area method (hereinafter referred to as the EA method) is proposed, which assumes that the ideal crack pixel will be the darkest in the image, and determines the boundary of the crack by comparing the gray difference between the crack region and the background. Due to the complexity of the optical imaging system, the grayscale of the entire actual crack region is absorbed by the crack region in the image and the surrounding adjacent pixels (transition regions). In addition, the EA method takes into account the effects of uneven lighting and background color on crack measurements.

Please refer to FIG. 5 again, the specific implementation process of the EA method is as follows:
1. Convert the image of the crack region identified from the captured image of the scanned surface to a single-channel grayscale crack image.
2. Converting the single-channel grayscale crack image into a three-dimensional grayscale image, considering the influence of uneven illumination and background color on crack measurement, the background light intensity at the crack pixels in the three-dimensional grayscale image is fitted by using the RANSAC algorithm to obtain the background image.
3. Subtract the single-channel grayscale image from the background image to obtain a background crack image. The resulting background image can be denoised to reduce the impact of background noise.
4. Use the equivalent area principle to calculate the subpixel crack width of each pixel row in the background crack reduction image.

Firstly, the EA method is applied to the sub-background crack images, and the sub-pixel edge of the crack is determined by analyzing the grayscale distribution of the crack region. Subpixel edge localization refers to determining the position of the edge of an object with greater accuracy than the pixel resolution of an image. For example, in an image, if an edge spans multiple pixels, subpixel edge localization can determine the exact position of the edge between pixels, possibly at the 1.5th pixel, or at the 2.3rd pixel, etc. Subpixel edge localization is the basis of subpixel width measurement, and after determining the subpixel edge of the crack, the subpixel crack width of each pixel row can be obtained by measuring the distance between the subpixel edges of the crack.

In one embodiment, step S303 may also include determining the direction of the crack based on an image of the crack region. Knowing the direction of a crack can help to measure its width and length more precisely, as measurements can be made along the exact path of the crack rather than estimating it based solely on the distribution of pixels on the image. Direction of the crack can provide important information about crack propagation and structural stress distribution, helping to analyze the integrity of the structure and potential risk of failure. Accurate crack orientation data can improve the accuracy of crack width measurements, especially when using the EA method with sub-pixel accuracy, ensuring that the measurements are closer to the true value. In structural health monitoring, changes in direction of the crack can be used as an indicator of possible further deterioration of the structure, helpful for timely maintenance and repair measures. The automated crack detection and direction determination process reduces the need for manual measurements and increases the efficiency and speed of data processing. The ability to accurately determine the direction of a crack allows the measurement method to be adapted to a wide range of complex situations, including cracks of different shapes, sizes, and orientations.

In general, morphological methods can be used to process the image of the crack region to obtain crack skeleton to determine the direction of the crack. For example, the U-Net model can be used to detect the image of the crack region, and the crack pixels are marked as white and the background pixels as black to obtain a binary image. Then, by applying skeletonization techniques such as the Zhang-Suen algorithm (Zhang, T. Y., Suen, C. Y.: A fast parallel algorithm for thinning digital patterns. Comm. ACM, 27 (3): 236-239 (1984) DOI: 10.1145/357994.358023), the center line of the crack is extracted from the binary image to obtain the crack skeleton. Skeletonization is a technique in image processing that reduces the shape of an object to a skeleton with a single pixel width while retaining its main structural features. After the crack skeleton is obtained, the course of the skeleton is analyzed to determine the direction of the crack. This can be achieved by analyzing how the pixels on the skeleton are connected and arranged in the direction. In one embodiment, a curve-fitting method may also be used to more accurately determine the direction of extension of the crack.

In step S403, the pixel scale of the crack is determined:

First, a three-dimensional measurement model is established using a parallel laser line scanning device shown in FIG. 2A. This three-dimensional measurement model, also known as a three-dimensional laser camera measurement model, is used to characterize the relationship between the points on the optical plane where the laser fringe is located and its corresponding image pixel coordinates, as P(X_{c}, Y_{c}, Z_{c}) P′ (u, v) shown in FIG. 2A. This relationship is defined by employing geometric and triangular correlations, as described above in conjunction with the principle of measurement, where O-XYZ represents the world coordinate system, and O is the origin of the world coordinate system. Oc-XcYcZc represents the camera coordinate system, and Oc is the origin of the camera coordinate system, o-xy represents the image coordinate system, and o is the origin of the image coordinate system. Ouv-uv represents the pixel coordinate system, and Ouv is the origin of the pixel coordinate system. Point P is a point on the curve where the laser plane πp intersects with the surface of the measured object, and point P′ is the ideal projection point of point P on the image plane. Use the pinhole camera model to establish a positional relationship between the measurement point (P) and its image pixel (P′), as shown in Eq. (6):

$$s [ \begin{matrix}u \\ v \\ 1\end{matrix} ] = [ \begin{matrix}A & 0\end{matrix} ] [ \begin{matrix}{X}_{c} \\ {Y}_{c} \\ {Z}_{c} \\ 1\end{matrix} ] \qquad ( 6 )$$

where s represents the random zoom factor, and A is the 3×3 matrix of the parameters within the camera, including the effective focal length, camera image sensor size, and principal point. Eq. (6) indicates that any spatial point along the line O_{c} P can be projected onto the same pixel location (P′) in the image. In addition, the three-dimensional coordinates of the measurement point P satisfy the laser plane equation π_{P} expressed as follows in Eq. (7):

$${A}_{i} {X}_{c} + {B}_{i} {Y}_{c} + {C}_{i} {Z}_{c} + {D}_{i} = 0 \qquad ( 7 )$$

where, Aᵢ, Bᵢ, Cᵢ, Dᵢ are the physical coefficients that describe the plane equation of a single laser. When the camera parameters and laser plane parameters are determined, the simultaneous equations (6) and (7) can realize the calculation of the spatial three-dimensional coordinates of the measurement point P.

However, in actual scanning scenes, where the camera is often not parallel to the scanned surface and nonlinear distortion caused by camera manufacturing and mounting errors, the measurement reference plane needs to be restored for coordinate alignment in order to determine the pixel scale. Therefore, the process of determining the pixel scale also involves the recovery of the measurement reference plane using two sets of internal feature points identified by the laser baseline recovery algorithm. The main steps to determine the pixel scale of a crack are as follows:
1. Calibrate the system parameters of the device shown in FIG. 2A. System parameters include camera parameters (internal parameters and distortion coefficients) and laser plane parameters.
2. Extract the laser stripe image from the captured image of the scanned surface, and determine the center of the laser stripe (according to the laser stripe image as shown in FIG. 5). Thanks to the Steger method (Steger, C., Ulrich, M., & Wiedemann, C. (1998). Machine vision algorithms and applications. Wiley-VCH.) can achieve higher precision and robustness in the presence of noise and interference in the center of the subpixel laser band, therefore, in one embodiment, the Steger method is used to extract the centerline of the laser band, but other extraction algorithms, such as the extreme value method, the centroid method, the template matching method, etc., can also be used.
3. Laser baseline fitting. In one embodiment, the RANSAC method can be used to fit the laser baseline. The RANSAC method (Fischler, M. A., & Bolles, R. C. (1981). Random sample consensus: A paradigm for model fitting with applications to image analysis and automated cartography. Communications of the ACM, 24 (6), 381-395.) is known for its robust regression capabilities and is not affected by outliers. Considering that the target surface may be uneven, two laser baselines must be recovered to establish the target plane and thus precisely determine the pixel scale. The RANSAC method divides the dataset into internal points and outliers, and only fits the model to the internal points. In the process of determining the laser band center above, a batch of feature points are extracted from the laser fringe image, including undistorted laser spots, distorted laser spots, and noisy background points (represented as pixel areas with uneven light intensity distribution). By identifying the undistorted laser points as the internal points and the other feature points as outliers, RANSAC effectively mitigates the influence of the error feature points on the ideal model line. The RANSAC algorithm is performed twice to estimate two baselines from feature points. Firstly, the RANSAC is used to recover the first laser baseline. A residual threshold is set to identify points within the model. Then, by defining a distance limit from the first model, the feature points that belong to the first laser line are removed. Subsequently, RANSAC is invoked again with a cropped feature set to recover the second laser baseline. The two recovered laser baselines are actually two sets of undistorted laser spots.
4. Determine the target plane, and use Eq. (6) and Eq. (7) to establish the relationship between the pixel coordinates of any image and its corresponding spatial coordinates on the target plane. Two sets of three-dimensional laser point clouds can be generated using the calibrated system parameters and the above two sets of undistorted laser spots, and the parameters of the target plane equation can be determined according to the two sets of three-dimensional laser point clouds (the specific process can be referred to the Hong Kong short-term patent application 30094946, the contents of which are all included in this article by citation). The target plane established in this way effectively solves the problem of pixel distortion, and the pixel scale of any image region can be calculated according to the target plane.

In step S404, the actual crack width is determined based on the pixel scale of the crack, the subpixel crack width and the direction of the crack:

First, the sub-pixel crack width is located on each pixel row perpendicular to the direction of the crack according to the direction of the crack and the sub-pixel crack width of each pixel row. The actual crack width is then determined based on the sub-pixel width located on each pixel row perpendicular to the direction of the crack and pixel scale of the crack.

In another embodiment of the present application, a method for measuring the crack width using the parallel laser scanning device described above is also provided. The method mainly comprises: step 1) using a laser source to emit two beams of laser beams in parallel to each other to scan a surface containing cracks to be measured; step 2) using a camera to capture an image of the scanned surface which contains parallel lines of laser projection on the scanned surface and all cracks that may exist on the surface; step 3) the captured image of the scanned surface is processed to determine the crack width in the scanned surface. Wherein, in step 3) a pre-trained neural network is utilized to identify a crack region image from the image of the scanned surface, subpixel crack width and direction of the crack are determined according to the crack region image, then the laser fringe region image is identified from the image of the scanned surface, the pixel scale of the crack is determined according to the laser fringe image and the identified crack region, and finally the actual crack width is determined according to the pixel scale, the subpixel crack width and the direction of the crack. The above process can be referred to the steps described above in conjunction with FIG. 4, and will not be repeated here.

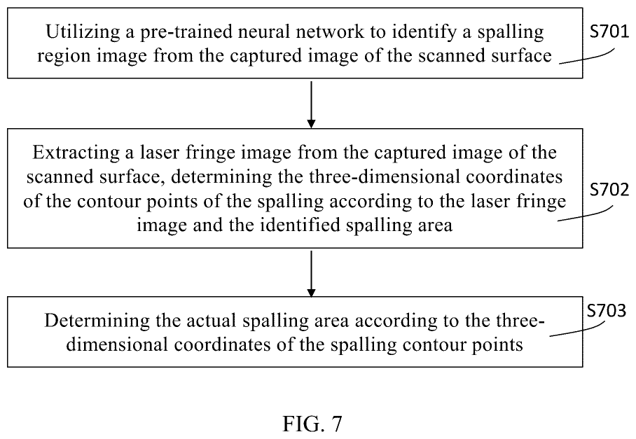

In another embodiment of the present application, the controller is configured to perform steps as shown in FIG. 7 to measure the spalling area, comprising: step S701, utilizing a pre-trained neural network to identify a spalling region image from the captured image of the scanned surface; step S702, extracting a laser fringe region image (subsequently referred to as a laser fringe image) from the captured image of the scanned surface, determining the three-dimensional coordinates of the contour points of the spalling according to the laser fringe image and the identified spalling area; step S703, determining the actual spalling area according to the three-dimensional coordinates of the contour points of the spalling. The above steps are described in detail below according to the process of measuring spalling shown in FIG. 7.

In step S701, the spalling region is identified.

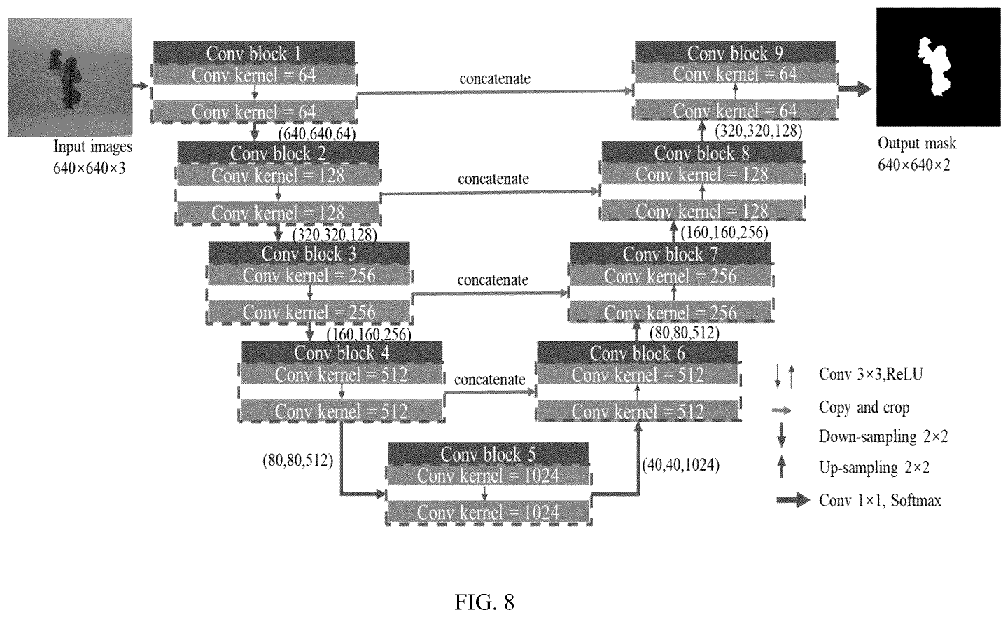

In one embodiment, a pre-trained neural network (which may be called an image segmentation network) is utilized to automatically segment or identify spalling region images from the captured images of the scanned surface. As shown in FIG. 8, in this embodiment, a U-Net network model is adopted to identify the spalling region image. In another embodiment, the backbone of the U-Net network model can also be replaced with ResNet-50.

In the present application, the use of the U-Net model for spalling identification is used as an example. The input of the U-Net model is an RGB spalling image, and the output is a semantically segmented binary image, where the spalled region is obtained by the U-Net algorithm. The architecture of the U-Net model is shown in FIG. 8 and includes an input layer, an output layer, and an intermediate layer. The middle layer consists of multiple convolution blocks (Conv blocks), each of which contains two convolutional layers. The left part in FIG. 8 represents the shrinking path, and the right part represents the expansion path. When the input RGB spalling image of size 512×512 passes through the shrinking path, the middle layer extracts the high-resolution features in the spalling image. After the output layer and the first convolutional layer of each convolution module, the RGB spalling image is compressed into a multi-channel feature map using 3×3 cores, and then the activation function is used to add nonlinearity to the neural network so that it can fit more nonlinear functions. In one implementation, the ReLU activation function a type of activation function commonly used to enhance nonlinear properties, is used. In order to reduce the number of parameters and the risk of overfitting in the network, the down-sampling operation is used as an intermediate process to connect each convolution module in the shrinkage path. Wherein, the maximum pooling operation is one of the methods of down-sampling, which has been shown to be able to preserve features well. After going through the encoding procedure, the features are passed to the extended path on the right side in FIG. 8. In contrast to the shrink path, the extended path progressively expands the calculated data shape to the original input image size to precisely restore and locate features. At the same time, the parallel feature maps from the shrinking path are passed directly to the extended path via the connection. The output layer is designed to be 1×1 in size and is used to convert the output image to binary of the same size and will store the category of each pixel into that pixel. In order to compress the pixel classification results in the form of normalization, the Softmax function is used in the output layer for normalization.

In previous studies on spalling semantic segmentation, several classical loss functions have been widely adopted, including cross-entropy loss function, dice loss function, focus loss function and their various combinations (Aslam, Y., Santhi, N., Ramasamy, N., Ramar, K.: Localization and segmentation of metal cracks using deep learning. J. Ambient Intell. Humaniz. Comput. 12, 4205-4213 (2021)). In addition, data imbalance is a problem in the peel identification task. Because the percentage of flaking pixels is sometimes small, it can cause the training weights to shift. In order to obtain the accuracy loss value in the training process and solve the problem of data imbalance, the combination of Generalized Dice Loss (GDL) and Cross-Entropy Loss (CEL) is selected as the loss function.

After processing by the U-Net algorithm, the spalling regions in each image can be identified and extracted. As described in step S401 above, the quality of the trained model is usually evaluated by three parameters: Precision, Recall, and IoU, which will not be repeated here.

In step S702, the three-dimensional coordinates of the contour points of the spalling are determined.

First, a three-dimensional measurement model is established using a parallel laser line scanning device shown in FIG. 2B. The three-dimensional measurement model is the same as the three-dimensional measurement model established in step S403 above, and the process of its establishment is not repeated herein.

In the actual scanning scene, the camera is often not parallel to the scanned surface and the nonlinear distortion caused by the camera manufacturing and mounting errors. In order to determine the three-dimensional coordinates of the contour points of the spalling, it is also necessary to restore the measurement reference plane for coordinate alignment. Therefore, the process of determining the three-dimensional coordinates of the contour points of the spalling also involves the recovery of the measurement reference plane using two sets of internal feature points identified by the laser baseline recovery algorithm. The main steps to determine the three-dimensional coordinates of the contour points of the spalling are as follows:
1. Calibrate the system parameters of the device shown in FIG. 2B. System parameters include camera parameters (internal parameters and distortion coefficients) and laser plane parameters.
2. Extract the laser stripe image from the captured image of the scanned surface, and determine the center of the laser strip according to the laser stripe image. Thanks to the Steger method (Steger, C., Ulrich, M., & Wiedemann, C. (1998). Machine vision algorithms and applications. Wiley-VCH.) can achieve higher precision and robustness in the presence of noise and interference in the center of the subpixel laser band, therefore, in one embodiment, the Steger method is used to extract the centerline of the laser band, but other extraction algorithms, such as the extreme value method, the centroid method, the template matching method, etc., can also be used.
3. Laser baseline fitting. In one embodiment, the RANSAC method can be used to fit a laser baseline. The RANSAC method (Fischler, M. A., & Bolles, R. C. (1981). Random sample consensus: A paradigm for model fitting with applications to image analysis and automated cartography. Communications of the ACM, 24 (6), 381-395.) is known for its robust regression capabilities and is not affected by outliers. Considering that the target surface may be uneven, two laser baselines must be recovered to establish the target plane and thus precisely determine the three-dimensional coordinates of the contour points of the spalling. The RANSAC method divides the dataset into internal points and outliers, and fits the model to only the internal points. In the process of determining the laser band center above, a batch of feature points are extracted from the laser fringe image, including undistorted laser spots, distorted laser spots, and noisy background points (represented as pixel areas with uneven light intensity distribution). By identifying the undistorted laser points as the internal points and the other feature points as outliers, RANSAC effectively mitigates the influence of the error feature points on the ideal model line. The RANSAC algorithm is performed twice to estimate two baselines from feature points. Firstly, the RANSAC is used to recover the first laser baseline. A residual threshold is set to identify points within the model. Then, by defining a distance limit from the first model, the feature points that belong to the first laser line are removed. Subsequently, RANSAC is invoked again with a cropped feature set to recover the second laser baseline. The two recovered laser baselines are actually two sets of undistorted laser spots.
4. Determine the target plane, and use Eq. (6) and Eq. (7) to establish the relationship between the pixel coordinates of any image and its corresponding spatial coordinates on the target plane. Two sets of three-dimensional laser point clouds can be generated by using the calibrated system parameters and the above two sets of undistorted laser spots, and the parameters of the target plane equation can be determined according to the two sets of three-dimensional laser point clouds. The target plane established in this way effectively solves the problem of pixel distortion, and the three-dimensional coordinates of any image region can be calculated according to the target plane.

In step S703, the spalling area is determined based on the three-dimensional coordinates of the contour points of the spalling.

The centroid of a three-dimensional polygon formed by the contour points of the spalling is calculated to represent the location of the spalling. If the camera's spatial attitude is known (for example, by calibration or SLAM), the absolute position of the spalling region in the world coordinate system can also be determined. Since all contour points are roughly on the same object plane, a coordinate transformation is applied to project these three-dimensional points onto a local two-dimensional plane. This normalization simplifies subsequent area calculations. The area of the enclosed region of the resulting two-dimensional polygon is then calculated using the Shoelace algorithm to determine the spalling area. The method is robust to both concave and convex geometries, which significantly improves the accuracy and reliability of spalling area estimation.

In another embodiment of the present application, a method for measuring the spalling area using the parallel laser scanning device described above is also provided. The method mainly comprises: step 1) using a laser source to emit two laser beams in parallel to each other to scan a surface containing the spalling to be measured; step 2) using a camera to capture an image of the scanned surface which contains parallel lines of laser projection on the scanned surface and any spalling that may exist on the surface; step 3) the captured image of the scanned surface is processed to determine the spalling region in the scanned surface. Wherein, the step 3) comprising utilizing a pre-trained neural network to identify a spalling region image from the image of the scanned surface, a laser fringe region image from the image of the scanned surface, determining the three-dimensional coordinates of the contour points of the spalling according to the laser fringe region image and the identified spalling region, and finally, determining the actual spalling area according to the three-dimensional coordinates of the contour points of the spalling. The above process can be referred to the steps described above in conjunction with FIG. 7, and will not be repeated here.

### Experiments and Analysis

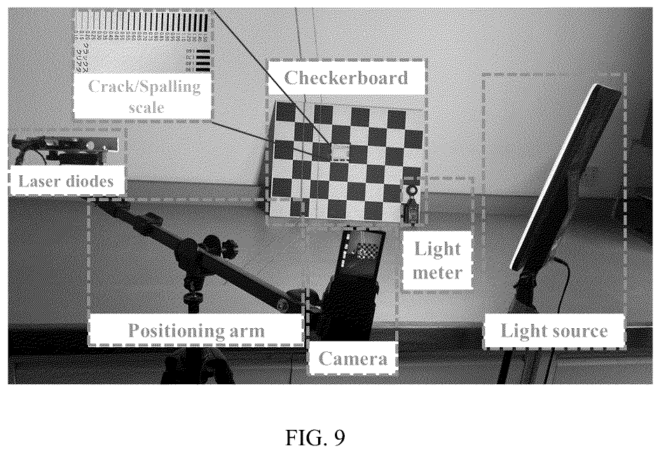

In order to evaluate the accuracy of measuring crack width or spalling area of the parallel laser line scanning device of the present application, and to analyze the impact of errors caused by equipment, algorithms, and environmental factors, a description of a number of experiments will be carried out step by step below. The artificial crack or spalling measurement scenario in the laboratory is shown in FIG. 9. The accuracy verification proposed in the present application is mainly divided into the following steps: first, the grid size on the calibration plate is measured from different distances by using the parallel laser line camera device proposed in the present application to evaluate the systematic error of pixel proportion or three-dimensional coordinate determination; Secondly, the U-Net segmentation algorithm and EA method are used to segment and measure the standard cracks on the crack scale or the standard spalling on the spalling scale to analyze the practicability of the algorithm. Python 3.8 is used to develop measurement programs and execute them on systems with Intel i7-6700, 3.40 GHz CPU, and 16 GB RAM.

TABLE 1

| Subassemblies | Specification | Parameters | |
|---|---|---|---|
| Camera | Model | Sony ZV-E10 | |
| | Sensor type | 23.5 × 15.6 mm (APS-C) CMOS | |
| | focal length | 16 | mm |
| | Resolution (H × V) | 6000 × 4000 | |
| Laser diodes | Model | PGL-LF-650-20 mW-DA10287 | |
| | | Red laser line emitters | |
| | Laser head size | Ø16 × 65 mm² | |
| | Bandwidth @3 m | 849.2 | μm |

| | | |
|---|---|---|
| | Projection angle | 60° |
| Power supply | Model | 3V PCB |

### 1) System Calibration

System calibration includes the deterministic system parameters in equations (8)-(9), including camera parameters and laser plane parameters. The camera calibration process is carried out by positioning the checkerboard in various positions and orientations. The camera intrinsic and distortion parameters are then determined using a camera calibrator toolbox based on least squares fitting and maximum likelihood estimation (Zhang, Z. Y.: A flexible new technique for camera calibration, IEEE Transactions on Pattern Analysis and Machine Intelligence, 22 (11), 1330-1334 (2000)). Thereafter, each laser plane is calibrated using a checkerboard with a single laser line. By adjusting the position and orientation of the chessboard, two sets of three-dimensional lines defining the laser plane are established. The calibration results can be viewed in Table 2 and these system parameters are valid within the measurement range of 1-2 m. It is important to note that these system parameters are confirmed prior to practical application, and the camera parameters (e.g. focal length) and measurement settings can be adjusted according to different situations (e.g. target measurement range).

TABLE 2

| | | | Left laser plane | Right laser plane |
|---|---|---|---|---|
| | | Camera distortion | equation | equation |
| | Camera intrinsic parameters | parameters | coefficients | coefficients |
| | (A) | (k₁, k₂, k₃, p₁, p₂) | (Aₗ, Bₗ, Cₗ, Dₗ) | (Aᵣ, Bᵣ, Cᵣ, Dᵣ) |
| Measuring distance: 1 m − 2 m | $[ \begin{matrix}4006.3 & 0.81 & 2071.3 \\ 0 & 4006.9 & 3 20.1 \\ 0 & 0 & 1\end{matrix} ]$ | (−0.047, 0.041, 0.000.003, 0.005) | (2.511, 0.355, −1,1647.1) | (2.476, 0.370, −1,1460.5) |

### 2) Evaluation of the Accuracy of the Measuring Device

The measuring device provides a pixel scale for assessing the crack width. The accuracy of this scale is assessed by projecting a dual laser line onto a calibration plate with a 1.0 cm grid. This checkerboard is measured in various directions and at distances between 1 and 2 meters. Using a determined checkerboard plane equation and a calibrated camera model, the pixel coordinates of each grid point on the checkerboard can be converted to three-dimensional coordinates. This allows the actual distance between grid points to be calculated. These distances are compared to a known true value (1 cm) to assess accuracy. To simplify the calculation, the average pixel scale is used to determine the distance between adjacent grid points. Since there are multiple grids on the calibration plate, the average of these measurements is used for more reliable comparisons. The corner extraction algorithms used are very complex and precise, suggesting that any experimental errors largely represent the systematic inaccuracies inherent in the measurement device.

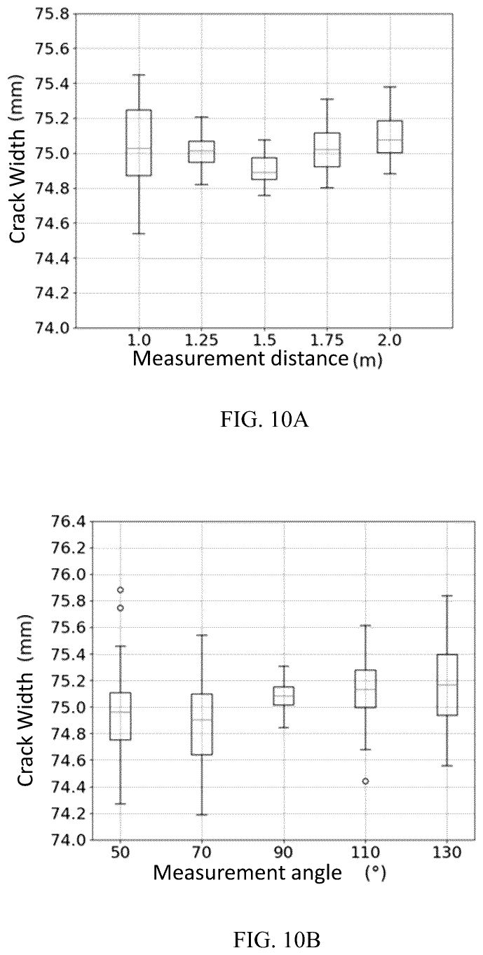

FIG. 10A shows the results of using vertical photogrammetry of the distance between adjacent corners in the range of 1.0 m to 2.0 m. The resulting average value is very close to the true value. At a minimum measuring distance of 1.0 m, the maximum error is 0.5 mm. This error occurs because the checkerboard corners are 1 cm apart equivalent to about 65 pixels, which amplifies the effect of the average pixel scale on the results. For small cracks (less than 10 pixels wide), this level of error is considered acceptable. FIG. 10B shows the measurement of the distance between adjacent corners at different angles at a measurement distance of 2.0 m. Here, too, the average value is very close to the true value, although at larger measurement angles, the maximum error increases to 0.9 mm. This suggests that the angle has a greater effect on the measurement results than the distance, which may be due to the increased pixel distortion on the measurement plane, which exacerbates the effect of using the average pixel scale for length determination. This error is also considered acceptable for small cracks (less than 10 pixels wide). Overall, the average values measured at different measurement distances and angles are always close to the true values, demonstrating the reliability of the pixel proportionality determination algorithm and the measurement device.

### 3) Measure the Width of Artificial Cracks

the performance of crack width measurement is evaluated using crack scales in a laboratory setting. As shown in FIG. 9, the crack scale is fixed on the chessboard, which serves as the measurement target plane for crack measurement. The designed measuring device projects parallel laser lines close to the crack scale. One side of the board includes light sources capable of providing different light intensities, and a light intensity meter can accurately measure ambient light intensity. The artificially generated cracks of various widths are marked on the crack scale convenient for evaluation.

The complete process of sub-pixel crack width measurement of artificial cracks can be divided into three steps: (1) detection and skeletonization: U-Net is used to detect artificial cracks, and the crack pixels are identified as white and the background pixels are marked as black. Subsequently, the Zhang-Suen skeletonization algorithm is used to obtain the crack skeleton. The skeleton is then analyzed to determine the average inclination of the crack. (2) Background restoration and crack region analysis: The RANSAC algorithm is used to recover the background information near the crack region. The gray scale distribution map shows that the gray value in the crack region decreases, and the background gray plane is robustly fitted. A differential slit image is then generated. Using the single-line grayscale distribution, the equivalent area principle is applied to realize the sub-pixel accuracy measurement of crack width. (3) Laser line processing and pixel scale determination: the dual laser line image is processed, the laser center line is identified, and the target plane direction is determined, so as to determine the pixel scale of the crack region. Given that cracks make up only a small portion of the image, the average pixel scale of the crack region is used to quantify the crack width. By determining the subpixel crack width, direction of the crack, and pixel scale, the actual crack width can be accurately restored.

The use of U-Net for crack segmentation is discussed in detail below. To evaluate the performance of the width measurement using the EA method, the recorded artificial crack width is compared to a known true value. In addition, the measured crack width is evaluated based on the results obtained using the Canny algorithm, which applies specific mathematical criteria for edge detection and serves as a reference for verifying the crack width. The influence of various factors such as measurement angle, distance, and lighting conditions on the measurement results is studied in detail.

FIGS. 11A to 11B show the measurement results of various artificial cracks using vertical photography at a measurement distance of 1.0 m. The pixel scale in these conditions is about 0.15 mm/pixel. In FIG. 11A, the horizontal axis represents the pixel rows of the 1.5 mm wide crack image, and the vertical axis shows the crack measurements for each row. These plots compare the results obtained with and without the EA method of image denoising with the results obtained with the Canny edge detection method. In FIG. 11B, the horizontal axis represents the pixel width of the measured crack, and the vertical axis shows the crack measurement for the entire crack.

As can be seen from FIG. 11A, the results of the Canny method are distinctly characterized by abrupt spikes and declines; These are especially noticeable when detecting oblique cracks due to pixel jumps at the edges of the cracks. Typically, for cracks with a width of 3 to 10 pixels (FIG. 11B), the actual crack width is within the range of values calculated by the Canny and the EA method. However, error analysis shows that the Canny method tends to be closer to the actual crack width than the EA method with an error of less than 0.075 mm (about 0.5 pixels) compared to the sub-pixel accuracy of the EA method. For cracks smaller than 3 pixels, the Canny method yields a result that differs significantly from the actual width, with an error of about 0.2 mm. the EA method, especially those that are not denoised, provide results that are closer to the true crack width. This difference is attributed to the 3×3 convolutional kernel used in Gaussian image denoising, which tends to obscure details in tiny cracks smaller than 3 pixels. In addition, careful selection of the internal parameters of the Canny algorithm is essential for crack edge identification when dealing with the very narrow crack widths detected. In contrast, the proposed EA method does not require parameter adjustments based on crack width and proves its effectiveness in measuring very fine crack widths. This comparison shows that the EA method can more accurately measure tiny cracks with a width of more than 2 pixels with an error of less than 0.5 pixels.

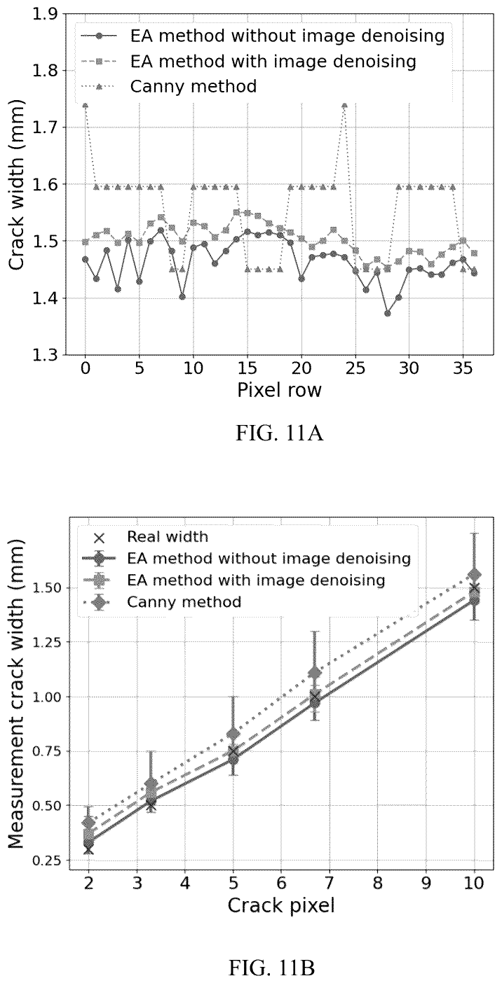

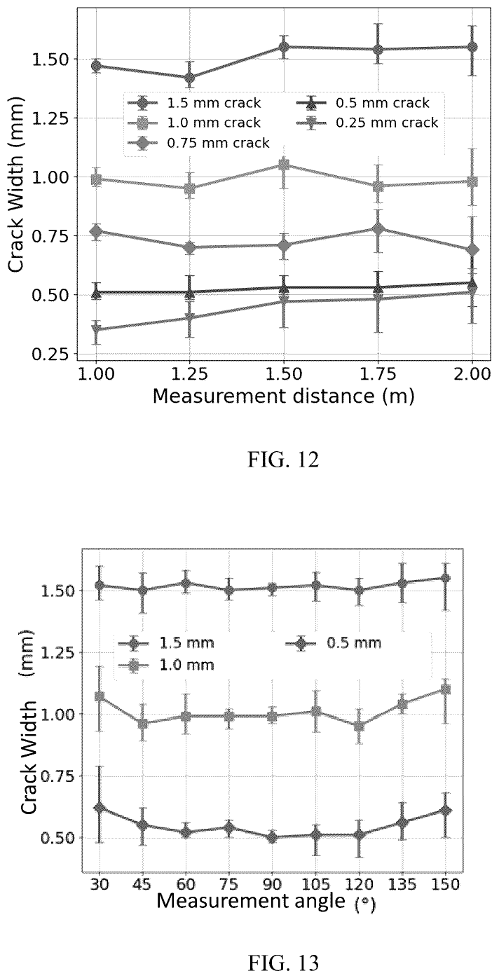

FIG. 12 shows the average measurement results and associated errors for artificial cracks with widths of 1.5 mm, 1.0 mm, 0.75 mm, 0.5 mm, and 0.25 mm over a measurement distance range of 1.0 m to 2.0 m using vertical photography. It is worth noting that all crack measurements are made using the denoising EA method, except for the 0.3 mm width crack measurement. The results show that for the cracks with widths of 1.5 mm, 1.0 mm, 0.75 mm and 0.5 mm, the measured average width is very close to the actual crack width and does not change significantly with the increase of distance. It is worth mentioning that at a measurement distance of 2 meters, the pixel scale is about 0.25 mm/pixel, which means that 0.5 mm crack width corresponds to about 2 pixels. This indicates that the measurement method can accurately measure cracks with a width of more than 2 pixels. However, as the measurement distance increases, the measurement error also increases, suggesting that larger measurement distances introduce more uncertainty to the results. At a distance of 2 meters, the maximum measurement error of different crack widths at the same crack scale reaches 0.12 mm (about 0.5 pixels). An important factor contributing to this increase in error is that the farther the distance, the greater the pixel scale, and the greater the uncertainty in the measurement. When measuring a 0.25 mm wide artificial crack, the error associated with the EA method increases with the measurement distance, resulting in a larger crack width and a greater deviation from the true value (maximum error of 0.2 mm). This indicates that the measurement method exaggerates the results of very fine cracks less than 2 pixels wide, thus magnifying the measurement error.

FIG. 13 shows the average results and errors of measurements for artificial cracks with widths of 1.5 mm, 1.0 mm, and 0.5 mm at a measuring distance of 1.5 m. Overall, measurements of various crack widths are often close to their actual values. However, as the measurement angle deviates by 90 degrees, the measurement error increases, resulting in a larger deviation from the true value. This effect is particularly noticeable for narrow 0.5 mm cracks. The increase in error when the angle deviates from the vertical direction can be attributed to the decrease in the number of pixels covering the crack width and the increased pixel distortion encountered in non-vertical photography. Both of these factors increase the uncertainty of the measurement results. The comparative data analysis shows that when the measurement angle is in the range of 45° to +135°, the crack measurement results are relatively stable, and the measurement error is kept below 0.12 mm.

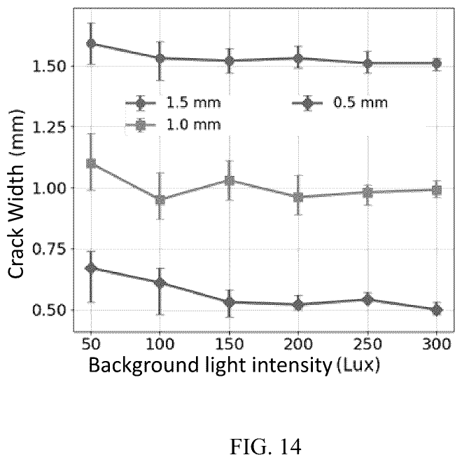

FIG. 14 investigates the effect of different lighting conditions on the measurement results of artificial cracks with widths of 1.5 mm, 1.0 mm, and 0.5 mm at a measurement distance of 1.5 m using vertical photography. The results show that at high background light intensities (>150 lux), the average measurement width of artificial cracks is usually close to their true value, with minimal measurement error. Conversely, when the background light intensity is below 150 lux, the average measurement width tends to deviate from the true value, and the measurement is usually larger than the actual width. This effect is especially pronounced in finer cracks (0.5 mm wide). Theoretically, as the intensity of background light decreases, the contrast between the crack and the surrounding environment decreases. The reduction of contrast leads to the small difference of gray value in the crack, which effectively reduces the effective height and area of the grayscale distribution of the crack.

As a result, the crack width trend measured by the EA method becomes complicated. When the reduction rate of area is less than the reduction rate of height, the measured crack width tends to be overestimated, and vice versa. Experiments have shown that decreasing the background light intensity significantly affects the measurement of finer cracks, thereby enlarging the measurement results. When the ambient light intensity is about 150 lux, the measurement results and errors are kept within an acceptable range (0.09 mm). In addition, excessively bright lighting conditions can hinder the precise positioning of the laser line and the accurate determination of the pixel scale, thus affecting the measurement results. Experimental results show that keeping ambient light intensity in the range of 150 to 2500 lux and measuring angles in the range of 50° to +130° results in a more stable measurement method.

The above laboratory results verify the effectiveness of the measurement method and lay a solid foundation for actual crack measurement. The applicability of these measurement angles, distances, and lighting conditions will be further validated in real-world crack measurement applications.

### 4) Measurement and Testing of Cracks in the Filed

The present invention is used to measure actual cracks in concrete walls in the laboratory and in the field. In addition, a crack measurement microscope is used as a reference standard to measure these actual cracks. To verify the accuracy of the crack measurement microscope, six sets of standard width cracks are measured: 0.03 mm, 0.05 mm, 0.75 mm, 1 mm, 1.5 mm, and 2 mm. The measurements are consistent, with an error kept to within 0.01 mm, demonstrating the reliability of the instrument for measuring actual crack widths. In addition, the U-Net model is trained on Google Colab using a single T4 GPU device.

The U-Net model is used for pixel-level concrete crack detection. In terms of data collection, images of concrete cracks are collected by parallel laser scanning. Concrete cracks come from a variety of locations, including residential buildings and parking lots, and can last anywhere from 30 to 50 years. To ensure model generalization and avoid overfitting, the light settings vary depending on the distance and angle at which the image is captured. A total of 350 images (each with a resolution of 6000×4000) are acquired, cropped into small pieces, and adjusted to 512×512 to train the U-Net model. Due to the difficult and time-consuming labeling process, only 450 images of the laser fringe region are segmented manually. To improve training performance and enrich the dataset, these images are expanded to 1000 images by image mirroring and random rotation. The dataset of a deep learning model is divided into a training set, a validation set, and a test set at a 6:2:2 ratio. Notably, the test images are specifically sourced from a different location than the training phase, emphasizing the goal of assessing the model's fitness.

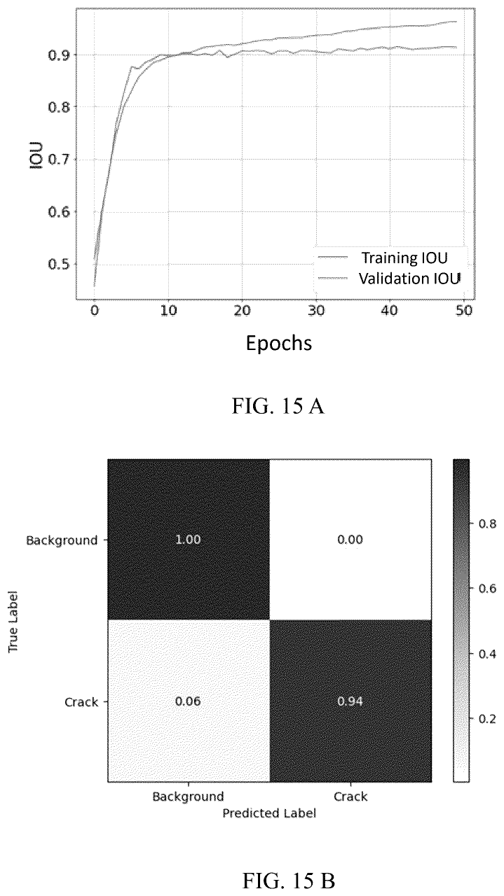

All relevant programs use the Adam optimization algorithm. FIG. 15A shows the training and validation intersection over union (IoU) curves for the U-Net model. The curves converge closely, indicating that the model has learned the relevant features for crack detection that are suitable for unseen data. FIG. 15B illustrates the performance of the U-Net model with a confusion matrix. The matrix effectively quantifies the ability of the model region to split the crack region and the background, showing an impressive IoU score of 0.94 for the crack region. The model exceeds the IoU benchmark of 0.9 and demonstrates extraordinary accuracy in identifying and segmenting even the smallest cracks.

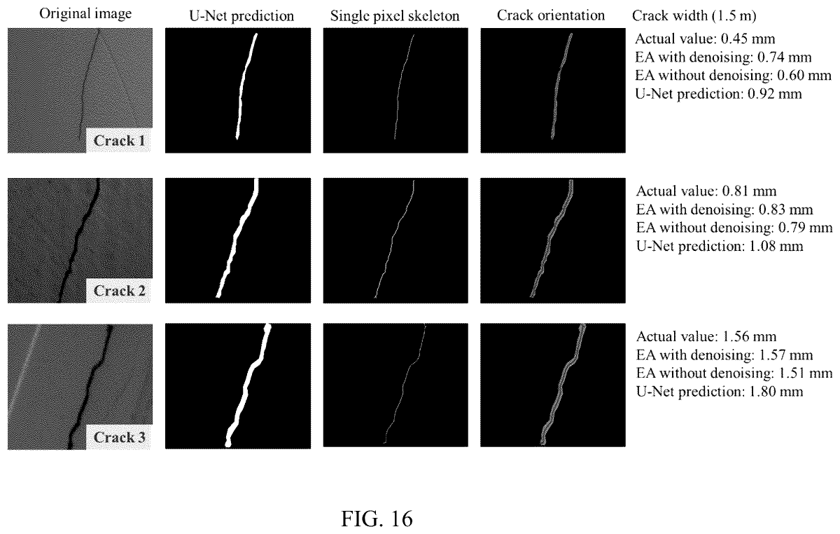

Three types of microcracks are selected to evaluate the performance of the proposed measurement method: Crack 1 with width of about 0.3-0.5 mm, Crack 2 with width of about 0.6-1.2 mm, Crack 3 with width of about 1.2-2.0 mm. The average crack width of each type of crack (Crack 1:0.45 mm, Crack 2:0.81 mm, Crack 3:1.57 mm) is determined using a crack measurement microscope. FIG. 16 shows the complete process of crack width measurement. First, the three types of actual cracks are segmented with U-Net. Then, the skeleton is generated using the detected crack pixels. Since the skeleton is pixel dependent, the curve fitting method is used to obtain the direction of the crack. Then the EA method is applied to obtain the subpixel crack width. At the same time, the laser line in the crack image is processed to calculate the crack scale, and finally the calculated average crack width is compared with the real value, and the crack width is calculated by using the crack boundary determined by U-Net, and the measurement result of the average crack width is shown on the right.

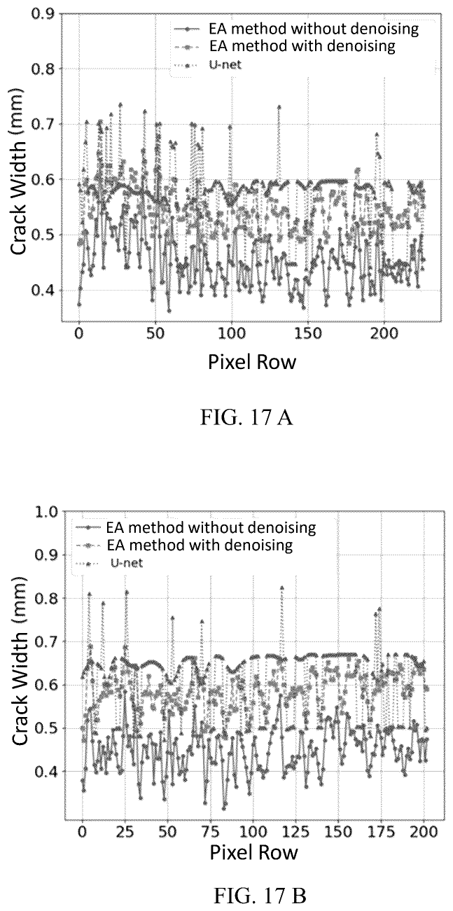

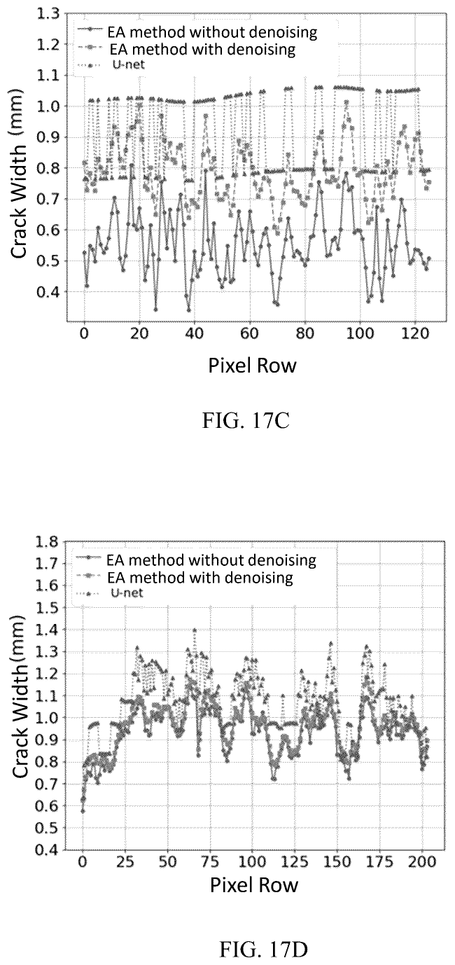

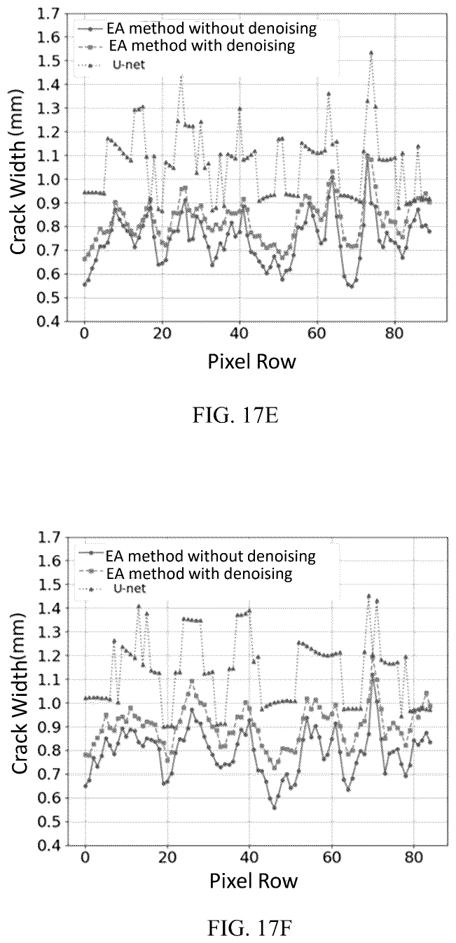

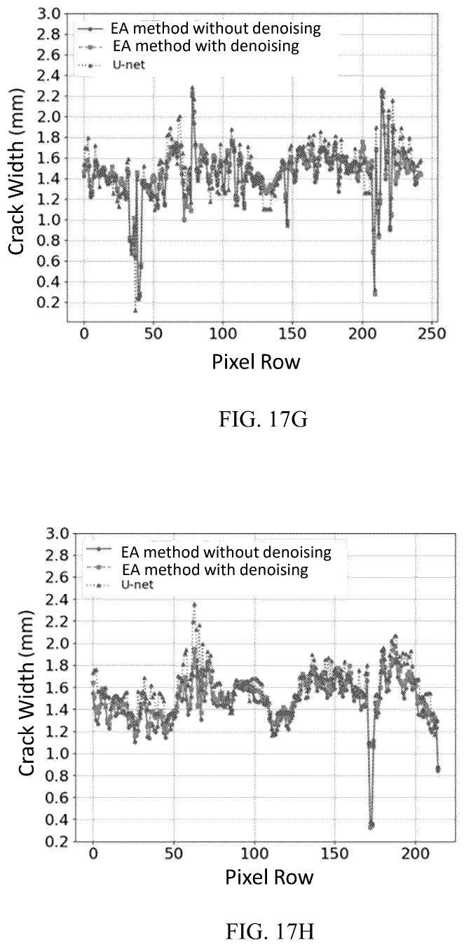

FIGS. 17A to 17I show the measurement results of three types of cracks (Crack 1, Crack 2, and Crack 3) at measurement distances of 1.0 m, 1.5 m, and 2.0 m using vertical photography. The horizontal axis of each plot represents the pixel rows of the crack image, while the vertical axis shows the measurements for each row. These plots compare the crack widths calculated using the EA method with and without image denoising and the results predicted by U-Net for the edge of the crack.

Observations from the results show that the predicted value of the U-Net is usually significantly larger than the true value, especially when the crack is small (Crack 1) and the measurement distance is longer. This is often due to the presence of grayscale transition zones at the edge of the crack, making it challenging to accurately delineate the exact edge of a narrow crack during U-Net prediction. In some cases, the EA method produces larger results than U-Net, probably because the crack edges are less pronounced at these locations and the transition zone is wider, resulting in less precise U-Net positioning. The accuracy of crack edge judgment in U-Net can be improved by more accurate crack annotation and increasing the number of training samples. From the measurements of the three crack types, the results using the denoising EA method are slightly larger than those without denoising consistent with the previous experimental conclusions. In addition, as the measurement distance increases, the abrupt change in the measured crack width becomes less pronounced, indicating smoother results due to lower resolution and less detail in the same crack region. The fluctuation range of crack width at different distances remains small, confirming the effectiveness of the 1-2 m distance measurement method. Given that the difference between denoising and non-denoising solutions is minimal, it is recommended to employ the EA method with image denoising in actual measurement activities to account for uncertainties in field conditions, such as uneven illumination and the color of the background around the crack.

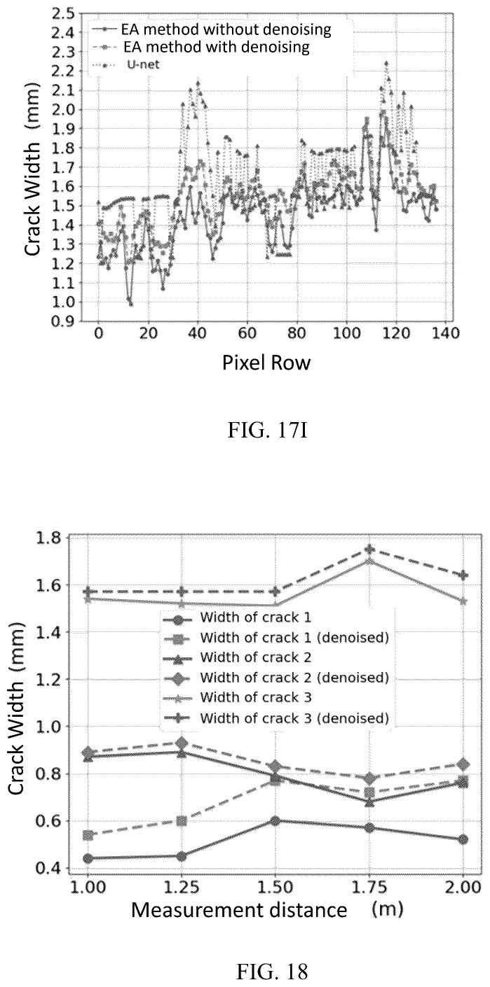

FIG. 18 shows the average crack width results measured for Crack 1, Crack 2, and Crack 3 over a measurement distance range of 1.0 m to 2.0 m using vertical photography. For all samples, the average width calculated by the two methods varies little with the measurement distance and is very close to the true value. The measurement error increases with distance, but the denoising EA method typically produces results that are closer to the true value, with maximum errors of 0.05 mm and 0.06 mm, respectively. For Crack 1, the deviation between the measured average crack width and the true value increases as the measured distance increases. Interestingly, the results of the EA method without image denoising are closer to the actual crack width with a maximum error of 0.09 mm compared to the results of denoising. This large difference compared to the denoising algorithm can be attributed to the 3×3 convolution kernel of the Gaussian image denoising algorithm, which can lose detail in tiny cracks with a pixel width of less than 3 pixels. Overall, the measurement error for all crack widths is kept within a small range (less than 0.1 mm), which indicates that the measurement algorithm can effectively capture the actual crack width. These results highlight the algorithm's powerful performance in real-world crack width measurements and highlight the subtle impact of denoising techniques on the accuracy of such measurements.

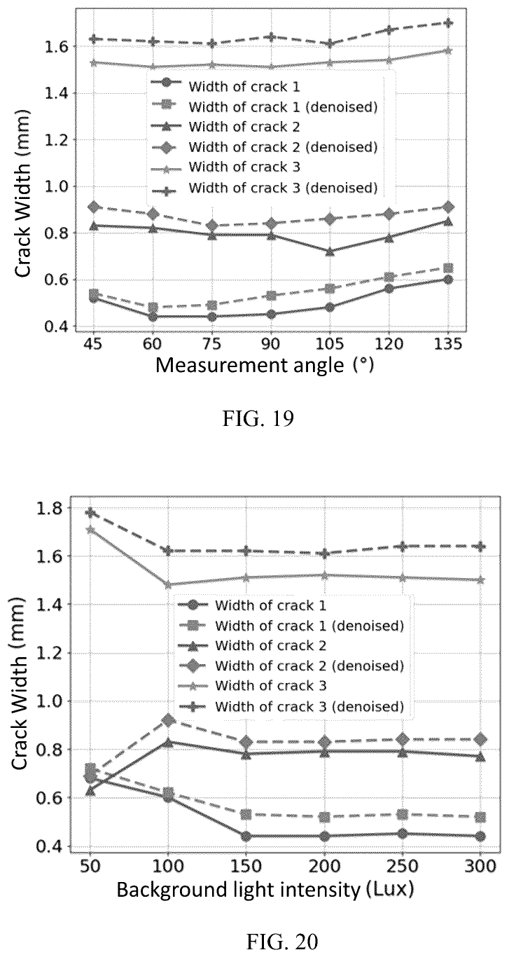

FIG. 19 shows the average crack width measurements for Crack 1, Crack 2, and Crack 3 at a distance of 1.5 m and an angle ranging from 45° to 135°. The results show that the average crack width does not change much with the measured distance. The measurements obtained using the EA method for image denoising are slightly larger than those obtained without denoising. Specifically, for Crack 1 at a larger oblique shooting angle, the measured width is slightly overestimated, with a maximum error of 0.1 mm. The increase in error is mainly due to the fact that the number of pixels on the crack width decreases as the angle increases, which leads to a decrease in measurement accuracy. The results show that the measurement method can accurately measure the crack width within the specified measurement angle range, and the error range is kept within 0.1 mm. This highlights the robustness and effectiveness of the measurement method in dealing with angle and distance changes during crack width assessment.

FIG. 20 shows the average crack width measurements of Crack 1, Crack 2, and Crack 3 at a measuring distance of 1.5 m at different ambient illuminations. The results show that the measurements of all three types of cracks remain stable with little variation when ambient light intensities exceed 150 lux, confirming the reliability of the measurement method under the recommended lighting conditions. However, at lower light conditions (less than 150 lux), the EA method calculated that all three types of cracks deviated significantly from the true values, suggesting that the system is not suitable for accurate crack measurement at such low light levels. Notably, the U Net model is capable of detecting cracks at both low and high optical densities.

The above experiments confirm that the proposed crack measurement method can accurately measure cracks, especially for small cracks. The optimal experimental conditions also proved to be practical and effective for crack measurement in the field. In addition, it is worth noting that, in addition to obtaining the crack width along the crack skeleton, the proposed method can derive the maximum width and length of the crack and other parameters that are essential for engineering inspection. This versatility increases the usefulness of the measurement method in a wide range of engineering applications.

In the present application, a practical and complete non-contact method for measuring the width of microcracks is proposed, which combines a laser camera system with an advanced image processing algorithm. At the heart of the effectiveness of this measurement method is a triangulation-based parallel laser line scanning device and a new image processing algorithm that accurately extracts pixel scale data from the laser strip, thereby minimizing the pixel distortion problems common in non-vertical photography. The U-Net algorithm is used for automatic crack detection and the EA method for accurate sub-pixel width calculation, which significantly improves the measurement accuracy. Through comprehensive laboratory and field testing, the present application has been shown to outperform traditional crack measurement techniques, consistently exhibiting small variations in error. It can automatically detect and measure cracks with a width greater than two pixels, achieving high accuracy under real-world conditions with a minimum difference of less than 0.5 pixels. The optimal parameters for its application include measuring angles between 45° and 135° and lighting conditions from 150 to 2500 lux, which have been rigorously validated. These conditions provide the most reliable results and emphasize the robustness and applicability of this measurement method in a real-world environment. This advancement marks a major breakthrough in structural health monitoring, providing an efficient, effective tool for building condition assessment and potentially transforming structural maintenance practices.

### 5) Area Measurement of Flat Objects

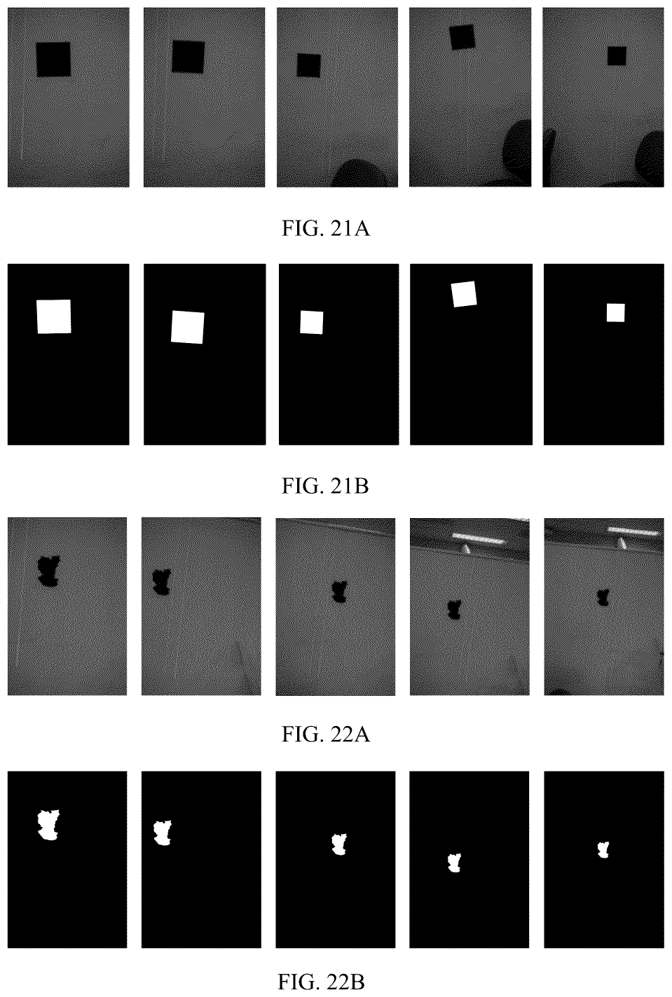

For accuracy verification, two black planar objects with a known surface area (Samples I and II) are used. There are several steps involved in the verification of the accuracy of the present application. First, a parallel laser line scanning device of the present application is used to project a laser line around the sample from various distances, angles, and lighting conditions. The scanned samples are then segmented by image binarization, including morphological denoising, erosion, and closure techniques. Subsequently, the boundary points of the body are extracted, and the conversion relationship between the boundary pixels of the object in the image and their corresponding three-dimensional coordinates is established by processing parallel laser lines. Finally, the Shoelace algorithm is used to calculate the actual area of the closed three-dimensional shape. FIGS. 21A and 21B show the raw image and segmentation results of sample I, respectively. FIGS. 22A and 22B show the raw image and segmentation results of Sample II, respectively.

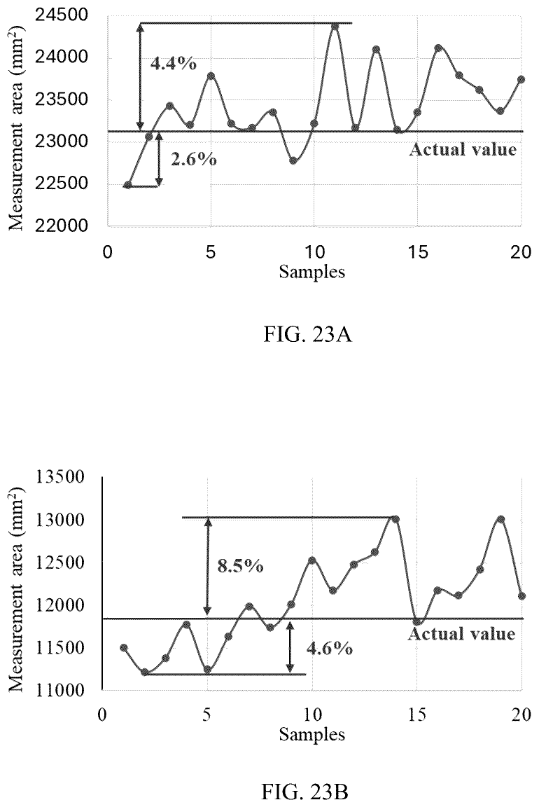

The area measurements for samples I and II are shown in FIGS. 23A and 23B, respectively. The known surface areas of Samples I and II are 23104 mm² and 11905 mm², respectively. As can be seen from the results of sample I in FIG. 23A, the measured area values at various angles, distances, and illumination intensities show small variations, all very close to the standard area, with an error of within 4.4%. This shows the validity and accuracy of the method of the present application in measuring the area of a conventional object. For sample II in FIG. 23b, the measured area values at different angles, distances, and lighting conditions also show slight variation, albeit slightly larger than sample I, with an error of within 8.5%. The increased variation may be due to the lower accuracy of boundary extraction for irregular edges; Still, these results demonstrate the effectiveness of measuring the area of irregular objects.

### 6) Measurement Test of Spalling in the Field

A parallel laser line scanning device of the present application is used to locate and measure the actual spalling on the ceiling soffit to assess the feasibility of the proposed method. The performance of spalling segmentation using the U-Net model is discussed below. In addition, the spalling area measurements are compared with those obtained in images captured at different viewing angles. U-Net model training takes place on Google Colab using a single T4 GPU device.

This U-Net model is developed for pixel-level concrete spalling detection. Images of concrete spalling are collected from a variety of old buildings, including residential buildings and parking lots between the ages of 30 and 50, by parallel laser scanning. To ensure model generalization and avoid overfitting, images are captured at different distances, angles, and lighting conditions. A total of 450 images, each with a resolution of 6000×4000 pixels, are cropped to smaller slices and adjusted to 640×640 pixels for training the U-Net model. Due to the complexity and timing of the marking, only 550 images of the laser fringe region are manually segmented. These images are augmented to 1300 images through mirroring and random rotation to improve the training effect. The dataset is split into a training set, a validation set, and a test set with a 6:2:2 ratio. It is important to note that the test images are from a different site than the one used in the training phase to effectively assess the adaptability of the model.

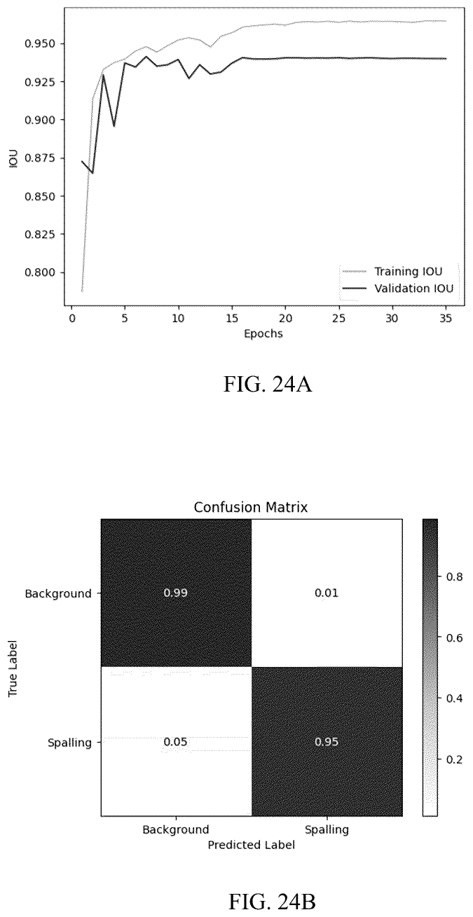

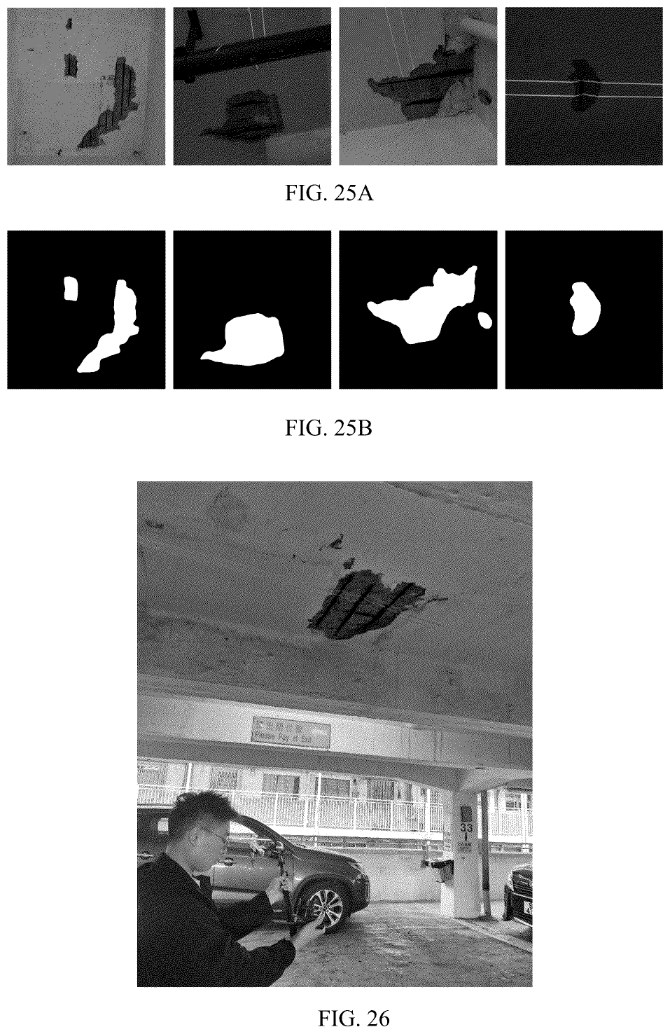

The Adam optimization algorithm is used throughout the training process. FIG. 24A shows the IoU curves on the training and validation sets during model training, which converge closely, indicating that the model learns efficiently from flaking detection features for invisible data. FIG. 24B shows a confusion matrix that quantifies the model's ability to distinguish between spalling regions and backgrounds, indicating an impressive IoU score of 0.94 for spalling regions. The model exceeds the benchmark IoU of 0.9, effectively dividing irregularly shaped spalling regions. FIGS. 25A and 25B show images and segmentation results of the spalling area sample, respectively, validating the accuracy of the U-Net model in spalling detection and segmentation, which is critical for subsequent area determination.

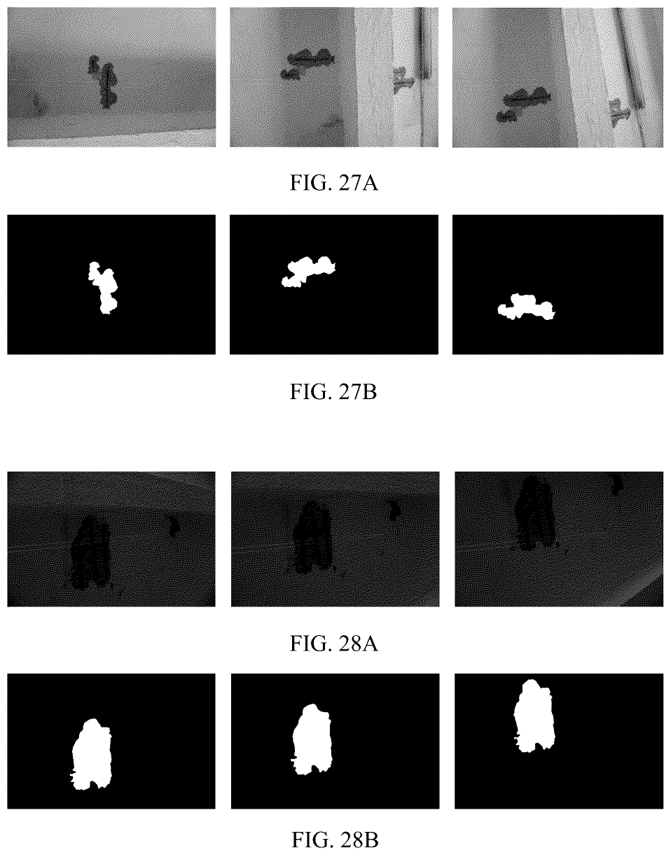

In order to evaluate the applicability of the algorithm of the present application in the area calculation, two spalling samples are selected for accuracy verification in the field scan scene (as shown in FIG. 26) measuring the soffit spalling of the ceiling of the parking lot. These samples are scanned under different ambient lighting conditions: Sample III scanned in brighter lighting (1200 lux) and Sample IV scanned in dimer lighting (150 lux). Accuracy verification involves projecting parallel laser lines around the spalled sample from different distances and angles. Subsequently, the U-Net segmentation algorithm segments the scanned samples and automatically extracts the contour points of the spalling. By processing parallel laser lines, a conversion from the contour pixel coordinates to their corresponding three-dimensional coordinates is established. Finally, the Shoelace algorithm calculates the actual spalling area. FIGS. 27A and 27B show the raw images and segmentation results of sample III, respectively. FIGS. 28a and 28b show the raw image and segmentation results of sample IV, respectively.

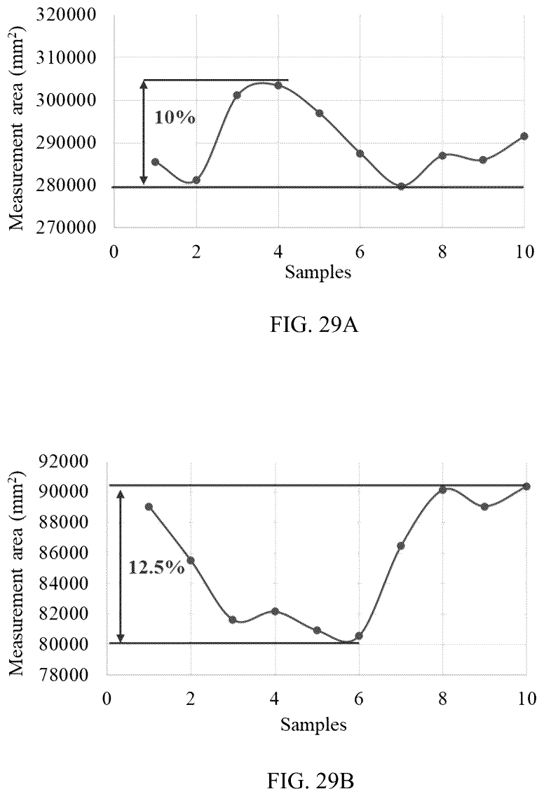

The area measurements for samples III and IV are shown in FIGS. 29A and 29B, respectively. Due to the lack of ground truth data, comparisons are limited to measurements of different scenarios. The results from Samples III and IV in FIGS. 29A and 29B show measurement variations of 10% for Sample III and 12.5% for Sample IV at various shooting angles, distances, and illumination intensities, which may be due to U-Net variations in boundary depiction under different conditions. Still, these measurement differences are acceptable for real-world engineering applications, such as estimating the extent of damage and repair effort. The consistency under different lighting and shooting conditions demonstrates the reliability and effectiveness of the method in the measurement of spalling area in situ.

This study proposes a practical and comprehensive non-contact method for automated localization and measurement of spalling, integrating a parallel laser line scanning device with advanced image processing algorithms. The handheld system captures spalling images in one go, then uses U-Net prediction and parallel laser line processing for accurate positioning. Spalling area and depth measurements outperform traditional techniques, with smaller variation in error, higher efficiency, and greater robustness over a wide range of environmental conditions.

As can be seen from the above experiments, the scanning device and measurement method provided in the embodiment of the present application for the measurement of defects in out-of-plane buildings, using parallel laser line scanning combined with an image processing algorithm based on machine learning, exhibits the ability to accurately measure defects, keeps the measurement angle between −45° and +45°, the distance is within 1-2 m, and the ambient lighting intensity is between 150 and 2500 lux, and can obtain stable and accurate area measurement with an error of less than 15%, as well as accurate crack width measurements with an error range of less than 0.5 mm, meet the requirements for detecting and measuring common building defects. The above evaluation of the scanning device and its image processing process under laboratory and field conditions shows that it is robust to different lighting conditions and complex backgrounds. Even under challenging lighting conditions, the scanning device employing the embodiment of the present application is capable of accurately detecting and quantifying building defects.

Although the present application has been described by means of preferred embodiments, the present application is not limited to the embodiments described herein, but also includes various alterations and variations made without departing from the scope of the present application.

What is claimed is:

1. A parallel laser line scanning device, comprising:
a laser source configured to emit two beams of laser light in parallel to each other to scan a surface;
a camera configured to collect an image of the scanned surface, and the camera comprising a lens and an image sensor;
a positioning rod, with the camera being arranged at a first end of the positioning rod and the laser source being arranged at a second end of the positioning rod; and
a controller configured to process an image of the scanned surface to obtain a pixel scale of a crack or three-dimensional coordinates of contour points of a spalling, and determine an actual crack width in the scanned surface according to the pixel scale of the crack, or determining an actual spalling area according to the three-dimensional coordinates of the contour points of the spalling.

2. The parallel laser line scanning device according to claim 1, wherein the laser source is a double-line laser diode or comprises two lasers.

3. The parallel laser line scanning device of claim 1, wherein the controller is further configured to perform the following steps:
utilizing a pre-trained neural network to identify a crack region image or a spalling region image from the image of the scanned surface;
determining subpixel crack width and direction of the crack according to the crack region image;
recognizing a laser fringe region image from the image of the scanned surface, determining the pixel scale of the crack or the three-dimensional coordinates of the contour points of the spalling according to the laser fringe region image and the identified crack or spalling region image;
determining the actual crack width according to the pixel scale of the crack, the subpixel crack width and the direction of the crack;
determining the actual spalling area according to the three-dimensional coordinates of the contour points of the spalling.

4. The parallel laser line scanning device of claim 3, wherein the neural network adopts a U-Net image segmentation network model.

5. The parallel laser line scanning device of claim 3, wherein determining the subpixel crack width according to the crack region image comprises:
converting the crack region image into a single-channel grayscale crack image;
converting the single-channel grayscale crack image into a three-dimensional grayscale image, and fitting background light intensity in the three-dimensional grayscale image to obtain a background image;
subtracting the single-channel grayscale image from the background image to obtain a background-reduced crack image; and
calculating subpixel crack width of each pixel row in the background-reduced crack image by using the equivalent area principle.

6. The parallel laser line scanning device according to claim 5, wherein
the background light intensity at the crack pixels in the three-dimensional grayscale image is fitted using RANSAC algorithm to obtain the background image.

7. The parallel laser line scanning device of claim 3, wherein determining the pixel scale of the crack or the three-dimensional coordinates of the contour points of the spalling according to the laser fringe region image and the identified crack region image or spalling region image comprises:
calibrating system parameters of the parallel laser line scanning device;
determining a laser band center according to the laser fringe region image;
fitting two parallel laser baselines according to the laser band center;
construct a three-dimensional measurement model representing relative positional relationship between laser spots on the scanned surface and image pixels in the image sensor;
generating two sets of laser point clouds corresponding to the two laser baselines in the laser fringe region image based on the three-dimensional measurement model, and determining a target plane based on the laser point clouds and the system parameters;
determining the three-dimensional coordinates of contour points of the crack or spalling according to the target plane and pixel coordinates of contour points of the identified crack or spalling; determining a pixel proportion of the crack according to the pixel coordinates and three-dimensional coordinates of the crack.

8. The parallel laser line scanning device of claim 7, wherein the laser band center is determined according to the image of the laser stripe area using the Steger method.

9. The parallel laser line scanning device of claim 7, wherein the RANSAC method is used to fit two parallel laser baselines according to the laser band center.

10. The parallel laser line scanning device of claim 7, wherein determining the three-dimensional coordinates of contour points of the crack or spalling according to the target plane and the pixel coordinates of contour points of the identified crack or spalling comprises:
calibrating system parameters of the parallel laser line scanning device; and
constructing a three-dimensional measurement model for representing a relative positional relationship between object points on the target plane and image pixels in the image sensor.

11. The parallel laser line scanning device of claim 3, wherein determining the direction of the crack according to the crack region image and the identified crack region comprises:
converting the crack region image into a binary image;
using a skeletonization technique to extract a center line of the crack from the binary image to obtain a crack skeleton; and
analyzing a direction of the skeleton according to the crack skeleton to determine the direction of the crack.

12. The parallel laser line scanning device according to claim 3, wherein determining the crack width according to the pixel scale, the subpixel crack width and the direction of the crack comprises:
locating a subpixel-level width of the crack on each pixel row perpendicular to the direction of the crack according to the direction of the crack and the subpixel crack width of each pixel row; and
determining the actual crack width according to the subpixel-level width of the crack located on each pixel row perpendicular to the direction of the crack and the pixel scale.

13. The parallel laser line scanning device according to claim 3, wherein determining the actual spalling area according to the three-dimensional coordinates of the contour points of the spalling comprises:
performing coordinate conversion of the three-dimensional coordinates of the contour points of the spalling to obtain a two-dimensional coordinate point set on the target plane;
sorting the set of two-dimensional coordinate points on the target plane according to a clockwise closed path; and
using the Soerace algorithm to process the set of two-dimensional coordinate points on the target plane to automatically adapt uneven contour and determine the actual spalling area.

14. A method for measuring crack width of a crack or spalling area of a spalling using the parallel laser scanning device of claim 1, comprising:
using the laser source to emit two laser beams in parallel to each other to scan a surface;
using the camera to capture an image of the scanned surface with a camera; and
using the controller to process the captured image of the scanned surface to determine the actual crack width or the actual spalling area in the scanned surface.

15. The method of claim 14, wherein processing the captured image of the scanned surface to determine the crack width in the scanned surface comprises:
using a pre-trained neural network to identify a crack region image from the image of the scanned surface;
identifying a laser fringe region image from the image of the scanned surface, determining the pixel scale of the crack according to the laser fringe region image and the identified crack region image;
determining subpixel crack width and direction of the crack according to the crack region image; and
determining the actual crack width according to the pixel scale, the subpixel crack width and the direction of the crack.

16. The method of claim 14, wherein processing the captured image of the scanned surface to determine the spalling area in the scanned surface comprises:
using a pre-trained neural network to identify the spalling region image from the image of the scanned surface;
identifying a laser fringe region image from the image of the scanned surface, determining three-dimensional coordinates of contour points of the spalling according to the laser fringe region image and the identified spalling region image; and
determining the actual spalling area according to the three-dimensional coordinates of the contour points of the spalling.
